(19) United States
(12) Patent Application Publication
(10) Pub. No.: US 2005/0074767 A1
(43) Pub. Date: Apr. 7, 2005

(54) Genes associated with obesity and methods for using the same

(76) Inventors: David Lewin, New Haven, CT (US); Timothy A. Stewart, San Francisco, CA (US)

Correspondence Address: MERCHANT & GOULD PC, P.O. BOX 2903, MINNEAPOLIS, MN, 55402-0903, US

(21) Appl. No.: 10/608,865

(22) Filed: Jun. 27, 2003

Related U.S. Application Data
(63) Continuation of application No. 09/691,439, filed on Oct. 18, 2000, now abandoned.
Application No. 09/691,439 is a continuation of application No. 60/160,246, filed on Oct. 19, 1999.

(51) Int. Cl. C12Q 1/68

(52) U.S. Cl. 435/6

(57) ABSTRACT

The instant invention is concerned with genes associated with obesity. Additionally, the invention provides methods of using the differential expression of these genes associated with obesity to diagnose and treat obesity, as well as to screen for compounds useful for treating obesity.

### RELATED U.S. APPLICATIONS

This application claims priority to U.S. Ser. No. 60/160,246, filed Oct. 19, 1999, which is incorporated herein by reference in its entirety.

### FIELD OF THE INVENTION

The invention relates generally to genes associated with obesity and methods for detectin and modulating obesity using such genes.

### BACKGROUND OF THE INVENTION

Obesity is a complex phenotype with highly variable causes and complexities. It is possible that obesity resulting from the loss of leptin function may have different qualities amenable to one therapeutic approach while being refractile to another. Obesity is the most prevalent metabolic disorder in the United States affecting on the order of 35% of adults at an estimated cost of 300,000 lives and $70 billion in direct and indirect costs. As an epidemic, it is growing due to the increase in the number of children who can be considered overweight or obese. Obesity is defined as an excess of body fat, and it frequently results in a significant impairment of health. Obesity results when adipocyte size or number in a person's body increases to levels that may result in one or more of a number of physical and psychological disorders. A normal-sized person has between 30 and 35 billion fat cells. When a person gains weight, these fat cells first increase in size and then in number. Obesity is influenced by genetic, metabolic, biochemical, psychological, and behavioral factors. As such, it is a complex disorder that must be addressed on several fronts to achieve lasting positive clinical outcome [1-3].

Obese individuals are prone to ailments including type II diabetes mellitus (NIDDM), hypertension, coronary heart disease, hypercholesterolemia, osteoarthritis, gallstones, cancers of the reproductive organs, and sleep apnea. Sleep apnea involves episodes of not breathing during sleep that correlate with higher incidence of stroke and heart attack, two other factors contributing to obesity-linked morbidity and mortality among the clinically obese [1, 2].

Several well-established obesity treatment modes ranging from non-pharmaceutical to pharmaceutical intervention are known. Non-pharmaceutical interventions include diet, exercise, psychiatric treatment, and surgical treatments to reduce food consumption or remove fat such as liposuction. Appetite suppresants and energy expenditure or nutrient-modifying agents are the main focus of pharmacological intervention. Dexfenfluramine (Redux) and sibutramine (Meridia) and beta3-adrenergic agonists and orlistat (Xenical) are examples of such pharmacological interventions. [4].

### SUMMARY OF THE INVENTION

In one aspect, the invention involves a method of assessing the efficacy of an obesity treatment in a subject, wherein the method involves the steps of providing a test cell population capable of expressing one or more of the OB1-6 nucleic acid sequences; detecting the expression of one or more of these nucleic acid sequences; comparing the expression to that of the nucleic acid sequences in a reference cell population whose obesity stage is known; and identifying a difference in expression level, if present, between the test cell population and the reference cell population. In various embodiments, the subject can be a mammal, or, more preferably, a human. In still other embodiments, the method involves the comparison of two; four, six, or more of the OB:1-6 nucleic acid sequences. In other embodiments, the test cell population can be provided in vitro, ex vivo from a mammalian subject, or in vivo in a mammalian subject.

In another aspect, the invention involves a method of identifying a test therapeutic agent for treating obesity in a subject involving the steps of providing a test cell population capable of expressing one or more of the OB1-6 nucleic acid sequences; contacting the test cell population with the test therapeutic agent; detecting the expression of one or more of these nucleic acid sequences; comparing the expression to that of the nucleic acid sequences in a reference cell population whose obesity stage is known; and identifying a difference in expression level, if present, between the test cell population and the reference cell population. In different embodiments, the subject may be a mammal or, more preferably, a human. Additionally, the test therapeutic agent may be either a known anti-obesity agent or an unknown anti-obesity agent. When the test therapeutic agent is a know anti-obesity agent, it can be, for example, dexfenfluramine, sibutramine, a beta3-adrenergic agonist, and olistat.

In another aspect, the invention involves a method of identifying leptin-induced nucleic acid sequences wherein the method involves the steps of providing a test cell population from a subject whose obesity stage is known; measuring the expression of one or more nucleic acid sequences expressed by the test cell population; comparing the expression of the nucleic acid sequences in the test cell population with the expression in a reference cell population having the opposite obesity stage as the test cell population; determining which nucleic acids, if any, are differentially expressed in the test and reference cell populations; adding leptin to both populations; comparing the expression of the nucleic acid sequences after leptin treatment; and identifying a difference in expression levels, if present, between the two populations.

In a further aspect, the invention involves a method of identifying or determining the susceptibility to obesity in a subject. In this aspect, the method involves the steps of providing a test cell population capable of expressing one or more of the OB1-6 nucleic acid sequences; detecting the expression of one or more of these nucleic acid sequences; comparing the expression to that of the nucleic acid sequences in a reference cell population whose obesity stage is known; and identifying a difference in expression level, if present, between the test cell population and the reference cell population. The subject may be a mammal, or, more preferably, a human.

In an alternative aspect, the invention involves a method of treating obesity by administering an agent that modulates the expression or activity of one or more of the OB:1-6 nucleic acid sequences to a patient suffering from or at risk for developing obesity. This agent can be one that decreases the expression of one or more of OBs:1, 2, and 4-6. Alternatively, it can be one that increases the expression of OB:3. Additionally, the agent can be an antibody to a polypeptide encoded by the OB nucleic acid sequence, a peptide, a peptidomimetic, a small molecule, or another drug.

The invention also includes a kit containing one or more reagents for detecting two or more of the OB:1-6 nucleic acid sequences. Additionally, the invention involves an array of probe nucleic acids capable of detected two or more of the OB:1-6 nucleic acids.

Also included in the invention is an isolated nucleic acid molecule that this at least 75% identical to SEQ ID NO:1, or the complement of the nucleic acid sequence, as well as vectors and host cells containing this nucleic acid sequence.

In another aspect, this invention involves an isolated polypeptide that is at least 80% identical to a polypeptide encoded by the nucleic acid sequence of SEQ ID NO:1, or fragments, derivatives, analogs, or homologs thereof. Additionally, the invention also involves an antibody to the polypeptide, fragment, derivative, analog, and/or homolog.

In still further aspects, the invention involves pharmaceutical compositions containing either the isolated nucleic acid or the isolated polypeptide. Another aspect involves methods of detecting the presence of the nucleic acid and polypeptide.

Moreover, the polypeptides and nucleic acids of the invention can be used to treat obesity in a subject. When used to treat obesity, the expression status of the polypeptide and nucleic acids are regulated, i.e., up-regulated or down-regulated by leptin. Treatment of obesity may be in a mammal, preferably a human.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a diagram showing the results of the gene mapping of the OB6 gene.

FIG. 2 is a diagram depicting the proposed regulatory loop involving ACTH and leptin.

FIG. 3 is a diagram showing the QEA trace for one gene fragment corresponding to each of PC2 and POMC.

FIG. 4 is a diagram demonstrating the results of the real time quantitative PCR performed to characterize the transcript levels for a representative set of the genes (PC2 and POMC).

FIG. 5 is a diagram showing the translation analysis of the reverse complementary strand of OB6.

### DETAILED DESCRIPTION OF THE INVENTION

Animal models have provided strong evidence that genetic make-up is influential in determining the nature and extent of obesity. Although what is true in animals may not be true for humans, 40-80% of variation in body mass index (BMI, a measure of obesity correlating weight and height) can be attributed to genetic factors [2, 5]. While human obesity does not generally follow a Mendelian inheritance pattern [6], there are several rodent models which do [6, 7]. As human obesity is a complex trait, it is not surprising that single mutations in rodents might not be representative of causation in the majority of obese humans, though there are examples of humans with genetic lesions analogous to those found in rodents [8, 9]. In addition, animal models for complex phenotypes, such as hypertension and stroke are also obese. Thus, these animals may prove to be a more telling model for understanding the complexities of human obesity [10-12].

Several rodent models of obesity result in the inheritance of a single genetic lesion. Monogenetic obesity syndromes in mice that are well characterized but rarely, if ever, observed in humans include: obese (ob) and aberrant termination of the translation of the satiety factor leptin. Mutations of the leptin receptor result in the obese diabetic mouse (db), and Agouti (Ay) is a coat color mutant that is obese. Although normally only expressed in the skin, in the mutant animals this gene is ubiquitously expressed and may antagonize the binding of melanocyte stimulating hormone (MSH). MSH is derived from adrenocorticotropic hormone (ACTH), a major pituitary hormone that results from the proteolytic processing of the pro-hormone proopiomelanocortin (POMC). The fat phenotype is the consequence of a mutation in the hypothalamic pro-hormone converting enzyme carboxypeptidase E. The least well-characterized obese mouse mutant is tub. tub encodes a cytosolic protein that may influence the processing of hypothalamic neuropeptide hormones such as neuropeptide Y (NPY, an appetite stimulating hormone) and POMC [6, 7, 13, 14].

Recently, a POMC knockout mouse was reported that has a phenotype analogous to several mouse models for obesity, particularly that of A\hat{ }y. The POMC knockout has early onset obesity and has yellow hair color as well as adrenal insufficiency due to the apparent morphological absence of their adrenal gland. As there is no detectable corticosterone in these animals, and corticosterones increase food intake, it is surprising that these knockout mice are obese. The obese phenotype in these mice can be treated with alpha-MSH, a peptide hormone derived from POMC [15].

The obese (ob) mouse model, though monogenic, demonstrates complex phenotypes that are analagous to those observed in humans suffering from metabolic disorders that are either causative or the result of obesity. Ob mice (ob/ob) are animals deficient in leptin that have a profoundly obese phenotype that is responsive to treatment with leptin. The lean (ob/+ or +/+) siblings of ob/ob mice are non-responsive to leptin treatment. The obesity observed in this mouse model also manifests physiological consequences analagous to those observed in humans. Though obesity due to leptin deficiency is very rare in humans, it has been observed, and the morbidity is analogous to that recorded for the ob mouse. The leptin deficiency in ob mice leads to extreme early onset obesity. This model of obesity may provide a means by which to study the endocrine, signaling, and metabolic pathways involved in obesity. Identification of signaling molecules modulated by leptin may give clues to the regulation of leptin expression, perhaps identifying novel avenues for therapeutic intervention to treat obesity and obesity-induced disorders.

Other animal models, including fa/fa (fatty) rats, bear many similarities to both ob/ob and db/db mice. One difference, however, is that while fa/fa rats are very sensitive to cold, their capacity for non-shivering thermogenesis is normal. It is well established that thermogenesis and metabolism are closely coupled endocrinologically. Torpor, a condition analagous to hibernation and lethargy, seems to play a larger part in the maintenance of obesity in fa/fa rats than in the mice mutants. Further, several desert rodents, such as the spiny mouse, do not become obese in their natural habitats, but do become so when fed on standard laboratory feed [16].

The effects of leptin treatment upon gene expression in the pituitary of the obese mouse, ob/ob, and its heterozygous (ob/+), non-obese, siblings have been studied. The satiety factor leptin is a secreted hormone whose level in the serum is generally proportional to the amount of adipose tissue. However, this is not the exclusive source of this protein, since it can also be secreted by muscle and stomach under certain physiological conditions. Leptin is secreted by muscle and fat in response to elevated levels of glucosamine present in the blood after feeding [17]. The stomach secretes leptin in response to the hormone cholecystokinin, which is secreted by the pancreas in response to the presence of protein and fat in the intestinal tract [18, 19].

An absence of the hormone leptin leads to dramatic increases in appetite, food intake and adiposity. Leptin binds to its receptor, which is found in the hypothalamus as well as other regions of the brain. This binding results in modulation of food intake, energy expenditure, glucose metabolism, and fat metabolism. Leptin is also known to affect the immune competence of animals, and the ob/ob mouse is relatively difficult to propagate, suggesting effects on both the immune system[20] and reproductive behavior [21]. Leptin has also been observed to have angiogenic activity, suggesting that other cell types besides those in the brain may respond to leptin indirectly through the signaling of the leptin receptor, which is a member of the cytokine family of receptors that utilizes the JAK/STAT signaling pathway [22, 23].

Some of the effects that leptin has on the function(s) of peripheral organs involved in maintaining body composition are mediated through direct interaction of leptin with its receptor on the target tissue and some effects are indirectly mediated through secondary hormonal and neural pathways. Acute ventricular administration of leptin activates the STAT signaling pathway within relevant areas of the hypothalamus, induces expression of anorexigenic hormones and suppresses food intake (For reviews on the central effects of leptin see Elmquist et al., Nature Neuroscience 1(6):445-50 (1998) and Kalra et al., Endocrine Reviews 20(1):68-100 (1999)). In addition to direct central effects, leptin has peripheral effects that appear to be independent of the reduction in food intake. One of the first indications of this came from the observation that weight loss in obese rodents was in excess of that seen in mice pair fed to the leptin treated group. See Levin et al., Proc. Natl. Acad Sci USA 93(4): 1726-30 (1996). The peripheral effects of leptin appear to include both indirect effects mediated through hypothalamic changes and direct actions on the target tissues. In addition to the widely appreciated metabolic effects of leptin, this hormone also appears to impact fertility, angiogenesis, and the immune response. For recent reviews on the peripheral effects of leptin see references. See Cawthorne et al., Proceedings of the Nutrition Society 57(3):449-53 (1998) and Houseknecht et al. J. Animal Sci. 76(5):1405-20 (1998).

Additionally, there is ample evidence for the role of the pituitary, in concert with other endocrine organs such as the hypothalalmus and the adrenal glands (the hypothalamic-pituitary-adrenal axis, HPA), in the regulation of metabolism. Hypopituitarism is manifested by reduced or abolished secretion of one or more pituitary hormones. The consequences can be wide ranging and depend upon the nature of the hypopituitarism. In such cases where there is reduced or abolished secretion of ACTH, a peptide hormone derived from proopiomelanocortin (POMC), patients show symptoms including weight loss and anorexia. Hypersecretion of pituitary hormones, such as that observed with ACTH-secreting pituitary adenomas in Cushing's disease, include symptoms of obesity, hypertension and diabetes, as well as a number of other defects [24].

Until recently, no gene in humans had been found that was causative in the processes leading to obesity. Those that have been identified are analogous to those in some animal models for obesity and do not represent significant contributions to the obesity observed in human populations. Given the severity and prevalence, however, of disorders, including obesity, which affect body weight and body composition, there exists a great need for the systematic identification of such body weight disorder-causing genes. Addressing this need has been particularly difficult because obesity in humans is a complex, multifactorial, chronic disease developing into a phenotype that is affected by physiological, metabolic, cellular, molecular, social, and behavioral influences [2].

Few of the genes that are responsible for regulating body composition and the peripheral effects of leptin are known. A new gene profiling technology was used to characterize gene expression changes that occur in the pituitary, hypothalamus, fat, muscle and liver in response to both obesity and treatment with exogenous leptin. These differences were then overlaid to allow the identification of genes whose expression is altered by obesity and at least partially normalized by leptin treatment. Using this process six obesity and leptin responsive sequences expressed by the pituitary have been identified. The sequences encode polypeptides that are either secreted molecules, involved in processing secreted hormones, or appear to be involved in protein secretion. The results also indicate that ACTH may be involved in a regulatory loop involving leptin.

Gene expression changes have been used widely in the last decade to identify genes that may be relevant to physiological processes. These have included differential display (See Liang et al., Science 257 (5072):967-71 (1992)), RDA (representational difference analysis) (See Lisitsyn et al., Science 259 (5097):946-51 (1993)), SAGE (serial analysis of gene expression) (See Velculescu et al., Science 270 (5235): 4484-87 (1995)) and more recently array based technologies (See Schena et al., Science 270 (5235): 467-70 (1995)). For a recent review of these approaches see (Carulli et al., J. Cell. Biochem.—Supplement 30-31:286-96 (1998)). All of these techniques have proved useful but have limitations in terms of the ability to reproducibly detect small differences between samples, the number of different mRNA species that can be sampled, and the ability to make multiple independent comparisons.

Genes whose transcript levels varied between the cell lines were identified using GENECALLING™ differential expression analysis as described in U.S. Pat. No. 5,871,697 and in Shimkets et al., Nature Biotechnology 17:798-803 (1999). The contents of these patents and publications are incorporated herein by reference in their entirety.

An unlabeled oligonucleotide competition assay as described in Shimkets et al., Nature Biotechnology 17:198-803, was used to verify the identity of differentially expressed sequences.

The present invention is based on the identification of genes that are modulated in the opposite direction with leptin treatment in ob mice versus their lean siblings. For example, if a gene was up-regulated in an obese individual compared to a lean one, this gene would be of interest provided that leptin treatment resulted in the down-regulation of that gene. However, for the gene to still be of interest, there should also be no modulation in the lean mouse after treatment with leptin.

To identify differentially expressed genes, the following comparisons were made:
A) obese/leptin vs. obese/vehicle
B) lean/leptin vs. lean/vehicle
C) obese/vehicle vs. lean/vehicle

The vehicle used was phosphate buffered saline (“PBS”).

The expression patterns of interest were:
A:B:C modulated in the pattern Up-regulated:No change:Down-regulated
A: B: C modulated in the pattern Down-regulated:No change:Up-regulated

The results of this differential expression analysis are shown in Table 1. Of the bands identified, six nucleic acid sequences whose expression levels differed were chosen for further characterization. These sequences are referred to herein as OBs:1-6. A summary of the OB sequences analyzed is presented in Table 2.

One sequence (OB6) represents a novel gene. The other sequences identified have been previously described.

For a given OB sequence, its expression can be measured using any of the associated nucleic acid sequences in the methods described herein. For previously described sequences (OB:1-5), database accession numbers are provided. This information allows for one of ordinary skill in the art to deduce the information necessary for detecting and measuring the expression of the OB nucleic acid sequences.

TABLE 1
Results of Differential Expression Analysis

| | | | | | Differentially | | | |
|---|---|---|---|---|---|---|---|---|
| | | N-fold | Sub- | Total | Expressed | Locked | Bands | Genes |
| Set A | Set B | Thresholds | sequences | Bands | Bands | Bands | Confirmed | Confirmed |
| Ob/leptin | Ob/vehicle | +2/−2 | 69 | 22965 | 30 | 0 | 1 | 1 |
| Lean/leptin | Lean/vehicle | +2/−2 | 66 | 21481 | 9 | 0 | 0 | 0 |
| Ob/vehicle | Lean/vehicle | +2/−2 | 66 | 21415 | 118 | 3 | 11 | 6 |

TABLE 2
Nucleic Acid Sequences Discussed Herein

| | | OB | SEQ ID |
|---|---|---|---|
| Name | Acc # | Assignment | NO: |
| PC2/Kex2: Mouse | M55669 | 1 | |
| homologue of yeast | | | |
| precursor processing | | | |
| endoprotease Kex2 | | | |
| POMC: mouse | J00612 | 2 | |
| proopiomelanocortin | | | |
| (POMC) gene exon 3, | | | |
| complete cds | | | |
| Prolactin: mouse mRNA | X02892 | 3 | |
| for prolactin; | | | |
| Novel gene fragment | Cgmmr0y0158.4_2 | | |
| determined to be | | | |
| part of prolactin by FLC | | | |
| Alpha-Globin: mouse gene | V00714 | 4 | |
| for alpha-globin | | | |
| Mm HSGP25L2G 1: | Mm4060_0 | 5 | |
| probable homolog | | | |
| of X90872 H. sapiens | | | |
| mRNA for gp25L2 | | | |
| Novel Gene | Cgmmi0m0307.2_2 | 6 | SEQ ID |
| | | | NO: 1 |

Below follows additional discussion of the nucleic acid sequences whose expression is differentially expressed in response to leptin treatment.

OB1

OB1 was down-regulated in obese mice following leptin treatment. Kex2/PC2 was found to be modulated in response to leptin, a result that was confirmed by TaqMan analysis. Kex2 in the yeast Saccharomyces cerevisiae is a transmembrane, Ca2+-dependent serine protease of the subtillisin-like pro-protein convertase (SPC) family with specificity for cleavage after paired basic amino acids. At steady state, Kex2 is predominantly localized in late Golgi compartments and initiates the proteolytic maturation of pro-protein precursors that transit the distal secretory pathway. However, Kex2 localization is not static, and its itinerary apparently involves transiting out of the late Golgi and cycling back from post-Golgi endosomal compartments during its lifetime [28]. If this is the case for the mammalian homolog of Kex2/PC2, then this suggests that not only is this molecule a target for small molecule therapy, but also as a target for antibody therapeutics. Antibodies could reach the intracellular compartments and neutralize the protease at the site of POMC processing.

Therefore, the Kex2/PC2 protease that is responsible for the processing of POMC to its derived peptide hormones, especially ACTH, represents, at the very least, a qualified drug target. This gene is of a class against which small molecule therapeutics may be identified. It has been newly discovered to be modulated in response to obesity with its substrate in a manner that is counter to that observed in the POMC knockout mouse.

Many peptide hormones and neuropeptides are produced from larger, inactive precursors through endoproteolysis at sites usually marked by paired basic residues (primarily Lys-Arg and Arg-Arg), or occasionally by a monobasic residue (primarily Arg). Precursor cleavages at mono-arginyl and dibasic sites can be catalyzed by Kex2-like processing endoproteases. Nakayama et al., J. Biol. Chem. 267(23):16335-40 (1992). Two mammalian gene products, PC2 and PC3, have been proposed as candidate neuroendocrine-precursor processing enzymes based on the structural similarity of their catalytic domains to that of the yeast precursor-processing endoprotease Kex2. These two proteases can cleave proopiomelanocortin (POMC) in the secretory pathway of mammalian cells. Thomas et al., Proc. Natl. Acad. Sci USA 88(12):5297-301 (1991). See Nakayama et al., J. Biol. Chem. 267(23):16335-40 (1992)., Thomas et al., Proc. Natl. Acad. Sci USA 88(12):5297-301 (1991).

OB2

OB2 was down-regulated in response to leptin treatment. POMC is proteolytically processed by Kex2/PC2 to generate, among other peptide hormones, ACTH. Both POMC and Kex2/PC2 are co-modulated in response to leptin, a result that was confirmed by TaqMan analysis. POMC can be cleaved to yield adrenocorticotropin (ACTH) and -beta-lipotropin (LPH). Human beta-LPH can be cleaved to yield beta-endorphin-(1-31), beta-endorphin-(1-29), beta-endorphin-(1-28), gamma-LPH, and beta-melanocyte-stimulating hormone. Bovine N—POMC1-77 can be cleaved to yield gamma 3melanocyte-stimulating hormone. Azaryan et al., J. Biol. Chem. 268(16):11968-75 (1993). A POMC knockout results in an obese mouse with Agouti\hat{ }y-like phenotype, including yellow hair color. In addition, these mice have no adrenal gland and no corticosterone production. With the exception of the loss of the adrenal gland, this phenotype is reversible with administration of MSH, which is a product of POMC processing. See Azaryan et al., J. Biol. Chem. 268(16):11968-75 (1993)., Yaswen et al., Nat. Med. 5(9):1066-70 (1999).

The treatment of both male and female ob/ob mice with leptin stimulated hypothalamic POMC mRNA by about three-fold. These results suggested that an impairment in production, processing, or responsiveness to alpha-MSH, another product of POMC cleavage, may be a common feature of obesity. This also suggested that hypothalamic POMC neurons, which are stimulated by leptin, may constitute a link between leptin and the melanocortin system. Mizuno et al., Diabetes 47(2):294-97 (1998).

POMC prohormone convertase may be important as a small molecule drug target. POMC and Kex2/PC2 are co-modulated in manner suggesting that down regulation in response to leptin of these two molecules results, in part or in whole, in a loss of the obese phenotype.

OB3

OB3 (Prolactin) expression level was found to be up-regulated in response to leptin treatment. This result was confirmed by TaqMan analysis. Prolactin (PRL) is a 198 amino acid peptide hormone that is secreted by the anterior pituitary. A 52 amino acid fragment of the UTR was identified as modulated by the addition of leptin. Full length cloning determined that this fragment is associated with PRL. PRL is present during pregnancy, but its effect is blunted by the actions of other hormones. PRL stimulates lactation in response to the decrease of estrogen and progesterone after birth. Hyperprolactinemia in humans leads to hypergonadism. In women, hyperprolactinemia effects ovulation and ultimately results in infertility. Similarly, hyperprolactinemia in men leads to infertility brought on by decreased testosterone levels as result of decreased spermatogenesis. See Aron, D., J. Findling, and J. Tyrell, Hypothalamus & Pituitary, in Basic & Clinical Endocrinology, F. Greenspan and G. Strewler, Editors. 1997, Appleton & Lange: Stamford, Conn. p. 108-9.

Serum leptin levels were negatively correlated with prolactin in one study of lactating women. UI: 97176720. Another study using mice found that prolactin secretion greatly increased in a dose-related manner, but only with leptin concentrations of 10⁻⁷ to 10⁻⁵ M. Yu et al., Proc. Natl. Acad. Sci. USA 94(3):1023-28 (1997).

Prolactin is important for fertility. A recent publication assoicated “prolactin AND fertility AND leptin”. (See http://www.ncbi.nlm.nih.gov/entrez/query.fcgi?cmd=Retrieve&db=PubMed&list_uids=11014193&dopt=Abstract). The ob mouse is generally not very fertile. Thus, leptin's affect on prolactin may be responsible in part for the recovery of fertility observed in leptin-treated ob mice. (See http://www.ncbi.nlm.nih.gov/entrez/query.fcgi?cmd=Retrieve&db=PubMed&list_uids=9048626&dopt=Abstract; and http://www.ncbi.nlm.nih.gov/entrez/query.fcgi?cmd=Retrieve&db=PubMed&list_uids=10754484&dopt—Abstract). Therefore, prolactin may have utility in the area of fertility as well as metabolic disease applications.

OB4

OB4 was down-regulated in response to leptin treatment. Alpha-Globin is one of the two types of polypeptide chains in hemoglobin. Both alpha and beta globins are negatively regulated by glucocorticoids. Alpha chains build early embryonic hemoglobin gower-2 (with epsilon chains), fetal hemoglobin F (with gamma chains), and adult hemoglobin A (with beta chains).

OB5

OB5 was down-regulated in response to leptin treatment. Mm_HSGP25LG2G_{—}1 is a contig in excess of 1600 base pairs that is 75% identical to X90872H. sapiens mRNA for gp25L2 (GenBank Accession No. X90872). This contig has a 227 amino acid open reading frame (“ORF”) that is 78% identical and 85% similar to gp25L2. ORF has SS and may also have a cis-Golgi retention signal. However, there is also evidence of its expression on the cell surface. Additionally, there is some evidence that this molecule is a resident of the endoplasmic reticulum, perhaps a component of the translocon. See Holthius et al., J. Cell Sci. 108(Pt. 10):3295-305 (1995), Keuhn et al., Nature 391(6663):187-90 (1998)., Dominguez et al., J. Cell Biol. 140(4):751-65 (1998). See also (http:lwww.ncbi.nlm.nih.gov/entrez/query.fcgi?cmd=Retrieve&db=PubMed&list_uids=7492314&dopt=Abstract).

OB6

OB6 expression was down-regulated in obese mice following leptin treatment. This non-coding expressed mRNA identified as OB6 was followed up due to its potential role as a regulator of expression for another gene. One ORF contains weak conservation with CXC chemokines. Untranslated, polyadenylated RNAs may play a regulatory role in expression. An example of this is the H19/IGFII interaction, where H19 regulates the expression of IGFII (maternal imprinting). H19 and IGFII are less than one kb apart in genomic DNA. OB6 could not be extended by SeqExtend, and there were no hits with BLASTX. In addition, there was no significant BLASTN result, and GNE FLC of this gene gives no significant ORF.

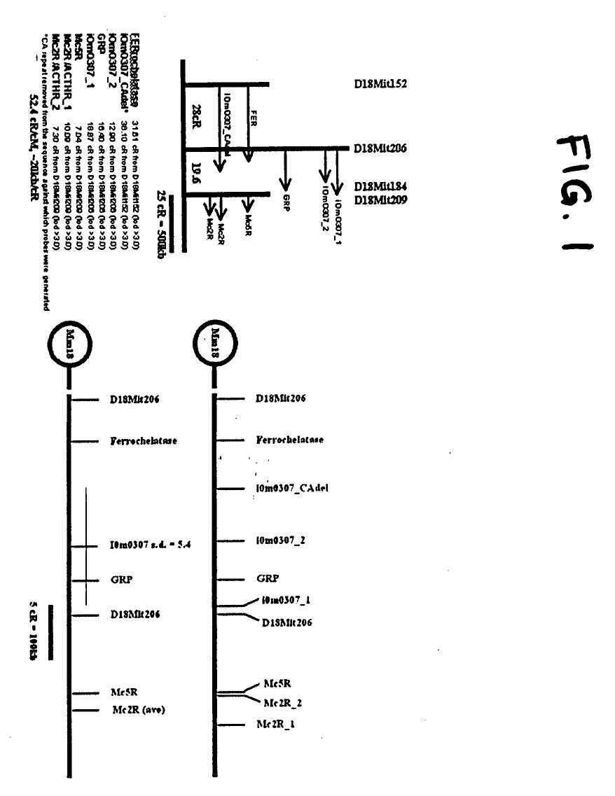

OB6 was mapped (FIG. 1) to determine whether it was in close proximity to genes that may play a role in metabolism. OB6 was found to be located near a cluster of melanocortin receptors [26] as well as nearest (as close as 100 kb) to gastrin-releasing peptide/bombesin. GRP/bombesin has been implicated in cellular proliferation [27]. It is possible that if GRP/bombesin is being regulated by OB6 and that OB6 has something to do with the proliferation of adipocytes.

Given the few numbers of genes found to be modulated in the pituitary, if OB6 regulates the expression of another gene, perhaps at the level of translation rather than at the level of transcription, determination of what gene is having its expression affected by OB6 may lead to the identification of an interesting gene whose expression may play a role in pituitary-mediated regulation of metabolic state.

The nucleic acid sequence of OB6 is presented below:

| | |
|---|---|
| TTATCTTGGCTTGGATTTGATTTTCTGTATCAGTAACTGACCATAGTGTTAAAGTATTAA | (SEQ ID NO: 1) |
| AATGGAGACCAGACCCAAAGCATAAAAAGGCACACAGTCATGGTCTTTCTCCTACGTG | |
| ACCTTAGCTTTGCATGATTTGAAAACAAAAAAGTTTTTTTAAAAAAGATTTATTTATTTA | |
| TTATATGTGATATAAACTACTTTAAATAGATTTGTATATTAAAGAAAACCAAAACAAAC | |
| TCAACCAATCCATGGCAGCCAAAATTTTATATAACTAGGGACTCTCCAATGGGAAGAG | |
| GCCAAATAAACAGCTGTGGAGCTGTAACCAATCACGTTGGCTTGGCGTTTATGCCTCCC | |
| TAATGAGTTAGTTCCCACCTGAAGTGCCTGGGCCACACAGGGGTTGGAGCTGCCCAGCA | |
| ACAACTGGTGTTTGCTCAGATACACTGTAACCCTTTAAGGTGCCTCAGCTGACACTTTA | |
| ACGTTAAGCAGTTACCTAATGTAGTACAGGTATCATAATCTAAGTCTTGAAGCTCATGA | |
| GGTTTATAACGCTGTTATTCTCACGAAAGTCACGTGACATAGCTTTCTATAACATGCTAT | |
| AGTAGTCCCCGTACCTCCAAGTGTTGTCTTTTTAGAGAGAATGATTTCCAGGGTCATTG | |
| AGGTCACTGAGGTAAGGAGGCCCCAGGTGAATGACCCACAGTGTCCTTGTAAAAAGAG | |
| ACACACACAGAGGGGCGATGAAATGCAGACACTGAATGAAGATGACCAACCATCTTCC | |
| ATCTCAGGAAGGACCAAACACTTCGGGAAGCTGTGAGAAGCCTATTTTAGAGCTCTAG | |
| AGAAGATCTACACACACACACACACACACACACACACACACACACACACACACACACG | |
| ACATCTGGCTGCCAGCAGTGTGAGACAGACAGACATTTCTGTTGTTTTGAGCCACTTAG | |
| TTGTAGTATTTTGTTAGAGCATCCCTAGGAAGCTAGAGCGCTCCTCTTACTCTACACCGG | |
| GTACATCTCAGGAGTCCCCCATGGATGGATGGTGGAAGCTGCAGACTATCAGCCCCTGT | |
| GTGTCCTGTTTTTCTGTATTCATTTATGCTTATGATAAAGTGTAACTTGTAAATTAGGCA | |
| AAGGAAGAAATAAACAACTACTAATAGTAAATAACTCACATTTAGAATGATTATAATAT | |
| ACTGTGTAACTTTGTAAGCAATATACTGCAATAAATGTTTTGCGACTGGGCCCTCCCTT | |

Leptin is known to alter hypothalamic gene expression, and gene expression within the pituitary is strongly influenced by proteins synthesized by the hypothalamus and delivered to the pituitary via hypothalamic-pituitary portal circulation. Thus, the effect of leptin on pituitary expression of POMC could be indirect. To address this, primary cultures of pituitary cells were established and treated with leptin. RNA was made from these cultures 24 hours after addition of the leptin, and POMC expression was monitored by real time quantitative PCR. The treatment of primary pituitary mouse pituitary cells with leptin for up to 24 hours did not alter the mRNA levels of either of prolactin or POMC. For the two genes tested, there was no evidence that leptin directly and acutely altered pituitary gene expression. The mRNA encoding the leptin receptor is present on pituitary cells, although the identity of the relevant cells is not known. It is possible that leptin directly alters the expression of the genes identified, but these changes were not detected under the culture and leptin exposure conditions used. As leptin has been shown to alter hypothalamic gene expression, and peptides derived from the hypothalamus have been shown to alter pituitary gene expression, it is also possible that the differences seen in this study are secondary to hypothalamic changes.

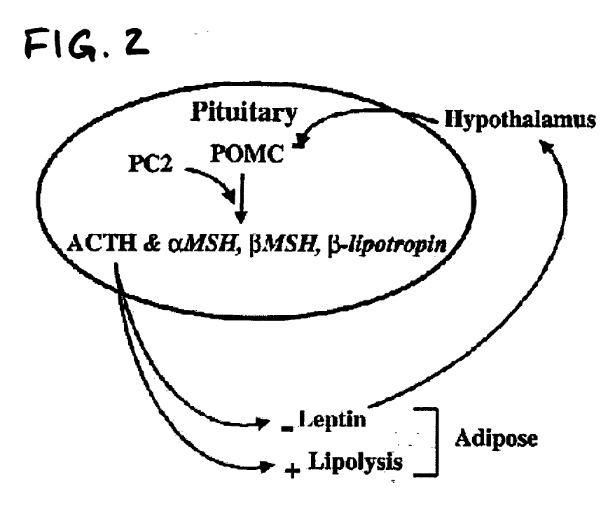

To test whether POMC/PC2 expression modulated the expression of leptin, thus defining a endocrine loop, adipocytes were treated with ACTH, a peptide derived from POMC after processing by PC2. Leptin expression by adipocytes was reduced both at the message and protein levels in response to ACTH. (See FIG. 2). However, this effect was not observed for other POMC-derived peptides, and this result is consistent with the negative correlation found between ACTH and leptin in vivo. The data support the concept of a regulatory loop involving leptin and ACTH. Specifically, an increase in ACTH will lead to a decrease in leptin and, in turn, the decrease in leptin will allow for the increased expression of ACTH. A possible contribution to this regulatory loop by prolactin was considered, but it was found that this peptide hormone had no effect on the metabolic status of adipocytes, as assayed by glucose uptake, glycerol release, or leptin secretion. One effect of obesity/leptin/prolactin may be in the area of fertility. All of the effects observed in vivo were limited to female animals.

Up to 2% of the genes expressed within a particular tissue are altered in response to obesity. However, only approximately 10% of these genes are returned toward normal after a one week treatment with leptin. Four known pituitary expressed genes (PC2, POMC, prolactin and HSGP25L2G_{—}1) have been shown to be both altered by obesity and at least partially normalized by leptin. Additionally, one novel gene (OB6) was also altered by obesity and at least partially normalized by leptin.

Other expression patterns of interest include those expression patterns that represent differences between ob/ob and lean mice, which are not corrected with leptin treatment. It is possible that such bands may belong to genes that are modulated as a secondary consequence of leptin deficiency. Although these are not responsive to leptin, they may be important for the maintenance of the obese phenotype as long as they are not simply a response to the stresses imposed by the obese phenotype. Additionally, these bands might be modulated in response to other compounds besides leptin. If this is the case, then they might represent novel avenues for the development of anti-obesity drugs, small molecule or antibody targets, as well as markers for the screening of patients or prognositics/diagnostics.

For example, a POMC knockout in mice results in a fat mouse with an agouti-like phenotype. [15] However, this is not the case in ob/ob mice, since these mice get slimmer in response to leptin treatment. In response to leptin, POMC is effectively down-regulated, which ultimately results in a thin mouse. This type of obesity may be fundamentally different from other kinds of obesity. Therefore, an examination of genes that are modulated in ob/ob vs. lean mice may also prove valuable.

General Screeening and Diagnostic Methods Using OB Sequences

Several of the herein disclosed methods relate to comparing the levels of expression of one or more OB nucleic acids in a test and reference cell populations. The sequence information disclosed herein, coupled with nucleic acid detection methods known in the art, allow for detection and comparison of the various OB transcripts. In some embodiments, the OB nucleic acids and polypeptide correspond to nucleic acids or polypeptides which include the various sequences (referenced by SEQ ID NOs) disclosed for each OB nucleic acid sequence.

In its various aspects and embodiments, the invention includes providing a test cell population which includes at least one cell that is capable of expressing one or more of the sequences OB 1-6, or any combination of OB sequences thereof. By “capable of expressing” is meant that the gene is present in an intact form in the cell and can be expressed. Expression of one, some, or all of the OB sequences is then detected, if present, and, preferably, measured. Using sequence information provided by the database entries for the known sequences, or the sequence information for the newly described sequence, expression of the OB sequences can be detected (if expressed) and measured using techniques well known to one of ordinary skill in the art. For example, sequences within the sequence database entries corresponding to OB sequences, or within the sequence disclosed herein, can be used to construct probes for detecting OB RNA sequences in, e.g., northern blot hybridization analyses or methods which specifically, and, preferably, quantitatively amplify specific nucleic acid sequences. As another example, the sequences can be used to construct primers for specifically amplifying the OB sequences in, e.g., amplification-based detection methods such as reverse-transcription based polymerase chain reaction. When alterations in gene expression are associated with gene amplification or deletion, sequence comparisons in test and reference populations can be made by comparing relative amounts of the examined DNA sequences in the test and reference cell populations.

For OB sequences whose polypeptide product is known, expression can be also measured at the protein level, i.e., by measuring the levels of polypeptides encoded by the gene products described herein. Such methods are well known in the art and include, e.g., immunoassays based on antibodies to proteins encoded by the genes.

Expression level of one or more of the OB sequences in the test cell population is then compared to expression levels of the sequences in one or more cells from a reference cell population. Expression of sequences in test and control populations of cells can be compared using any art-recognized method for comparing expression of nucleic acid sequences. For example, expression can be compared using GENECALLING™ methods as described in U.S. Pat. No. 5,871,697 and in Shimkets et al., Nat. Biotechnol. 17:798-803, both of which are incorporated herein by reference.

In various embodiments, the expression of 1, 2, 3, 4, 5, or all of the sequences represented by OB 1-6 are measured. If desired, expression of these sequences can be measured along with other sequences whose expression is known to be altered according to one of the herein described parameters or conditions.

The reference cell population includes one or more cells capable of expressing the measured OB sequences and for which the compared parameter is known, e.g., obesity stage. By “obesity stage” is meant that is known whether the reference cell is from an obese or a lean subject. For example, a subject with a positive obesity stage is obese whereas a subject with a negative obesity stage is lean.

Whether or not comparison of the gene expression profile in the test cell population to the reference cell population reveals the presence, or degree, of the measured parameter depends on the composition of the reference cell population. For example, if the reference cell population is composed of cells from an obese subject (i.e., a subject with a positive obesity stage), a similar gene expression level in the test cell population and a reference cell population indicates the test cell population has the same positive obesity stage. Likewise, a different gene expression level indicates that the test cell population has a negative obesity stage.

In various embodiments, an OB sequence in a test cell population is considered comparable in expression level to the expression level of the OB sequence in the reference cell population if its expression level varies within a factor of less than or equal to 2.0 fold from the level of the OB transcript in the reference cell population. In various embodiments, an OB sequence in a test cell population can be considered altered in levels of expression if its expression level varies from the reference cell population by more than 2.0 fold from the expression level of the corresponding OB sequence in the reference cell population.

If desired, comparison of differentially expressed sequences between a test cell population and a reference cell population can be done with respect to a control nucleic acid whose expression is independent of the parameter or condition being measured. Expression levels of the control nucleic acid in the test and reference nucleic acid can be used to normalize signal levels in the compared populations. Suitable control nucleic acids can readily be determined by one of ordinary skill in the art.

In some embodiments, the test cell population is compared to multiple reference cell populations. Each of the multiple reference populations may differ in the known parameter. Thus, a test cell population may be compared to a first reference cell population having a positive obesity stage as well as a second reference population having a negative obesity stage.

The test cell population can be any number of cells, i.e., one or more cells, and can be provided in vitro, in vivo, or ex vivo.

In other embodiments, the test cell population can be divided into two or more sub-populations. The sub-populations can be created by dividing the first population of cells to create as identical a sub-population as possible. This will be suitable, in, for example, in vitro or ex vivo screening methods. In some embodiments, various sub-populations can be exposed to a control agent, and/or a test agent, multiple test agents, or, e.g., varying dosages of one or multiple test agents administered together, or in various combinations.

Preferably, cells in the reference cell population are derived from a tissue type as similar as possible to test cell. In some embodiments, the control cell is derived from the same subject as the test cell, e.g., from a region proximal to the region of origin of the test cell. In other embodiments, the reference cell population is derived from a plurality of cells. For example, the reference cell population can be a database of expression patterns from previously tested cells for which one of the herein-described parameters or conditions (e.g., obesity stage, diagnostic, or therapeutic claims) is known.

The subject is preferably a mammal. The mammal can be, e.g., a human, non-human primate, mouse, rat, dog, cat, horse, or cow.

Identifying Leptin-Induced Nucleic Acid Sequences

Expression of some of the OB sequences described herein is induced by treatment with leptin. Thus, in one aspect, the invention provides a method of identifying leptin-induced nucleic acid sequences. By “leptin-induced” is meant that the expression of a particular nucleic acid sequence is modulated following treatment with leptin.

The method includes providing a test cell population from a subject whose obesity stage is known and measuring the expression of one or more nucleic acid sequences expressed by the test cell population. Next, the expression of the nucleic acid sequences in the test cell population is compared to the expression of the nucleic acid sequences in a reference cell population comprising at least one cell from a subject with the opposite obesity stage and determining which, if any, sequences are differentially expressed in the two populations. Then, both populations are treated with leptin. After such leptin treatment, the expression of the nucleic acid sequences in both populations are once again compared. Finally, the difference in expression levels between the two populations, if present, is identified. In this way, the leptin-induced nucleic acid sequences can be identified.

Methods of Diagnosing or Determining the Susceptibility to Obesity.

The invention further provides a method of diagnosing or determining the susceptibility to obesity. Obesity is diagnosed by examining the expression of one or more OB nucleic acid sequences from a test population of cells from a subject suspected of having the disorder.

Expression of one or more of the OB nucleic acid sequences, e.g. OBs:1-6 is measured in the test cell population and is compared to the expression of the sequences in the reference cell population. The reference cell population contains at least one cell from a subject not suffering from obesity. If the reference cell population contains cells that have a disorder, then a similarity in expression between OB sequences in the test population and the reference cell population indicates the subject is obese. A difference in expression between OB sequences in the test population and the reference cell population indicates that the subject is not obese.

The subject is preferably a mammal. The mammal can be, e.g., a human, non-human primate, mouse, rat, dog, cat, horse, or cow.

Methods of Treating Obesity

Also included in the invention is a method of treating, i.e., preventing or delaying the onset of obesity in a subject by administering to the subject an agent which modulates the expression or activity of one or more nucleic acids selected from the group consisting of OB. “Modulates” is meant to include increased or decreased expression or activity of the OB nucleic acids. Preferably, modulation results in alteration of the expression or activity of the OB genes or gene products in a subject to a level similar or identical to a subject not suffering from obesity.

The subject can be, e.g., a human, a rodent such as a mouse or rat, or a dog or cat.

In one aspect, the method of treatment involves the administration of an agent that decreases the expression of one or more of the nucleic acid sequences selected from the group consisting of OBs:1,2 and 4-6. Alternatively, the method can involve the administration of an agent that increases the expression of the OB3 nucleic acid sequence.

Suitable agents may include antibodies to polypeptides encoded by the particular OB nucleic acid sequence, a peptide, a peptidomimmetic, a small molecule, or other drugs.

The herein described OB nucleic acids, polypeptides, antibodies, agonists, and antagonists, when used therapeutically are referred to herein as “Therapeutics”. Methods of administration of Therapeutics include, but are not limited to, intradermal, intramuscular, intraperitoneal, intravenous, subcutaneous, intranasal, epidural, and oral routes. The Therapeutics of the present invention may be administered by any convenient route, for example by infusion or bolus injection, by absorption through epithelial or mucocutaneous linings (e.g., oral mucosal, rectal and intestinal mucosal, etc.) and may be administered together with other biologically-active agents. Administration can be systemic or local. In addition, it may be advantageous to administer the Therapeutic into the central nervous system by any suitable route, including intraventricular and intrathecal injection.

Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant. In a specific embodiment, administration may be by direct injection at the site (or former site) of a malignant tumor or neoplastic or pre-neoplastic tissue.

Various delivery systems are known and can be used to administer a Therapeutic of the present invention including, e.g.: (i) encapsulation in liposomes, microparticles, microcapsules; (ii) recombinant cells capable of expressing the Therapeutic; (iii) receptor-mediated endocytosis (See, e.g., Wu and Wu, 1987. J Biol Chem 262:4429-4432); (iv) construction of a Therapeutic nucleic acid as part of a retroviral or other vector, and the like. In one embodiment of the present invention, the Therapeutic may be delivered in a vesicle, in particular a liposome. In a liposome, the protein of the present invention is combined, in addition to other pharmaceutically acceptable carriers, with amphipathic agents such as lipids, which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,837,028; and U.S. Pat. No. 4,737,323, all of which are incorporated herein by reference. In yet another embodiment, the Therapeutic can be delivered in a controlled release system including, e.g.: a delivery pump (See, e.g., Saudek, et al., 1989. New Engl J Med 321:574 and a semi-permeable polymeric material (See, e.g., Howard, et al., 1989. J Neurosurg 71:105). Additionally, the controlled release system can be placed in proximity of the therapeutic target (e.g., the brain), thus requiring only a fraction of the systemic dose. See, e.g., Goodson, In: Medical Applications of Controlled Release 1984. (CRC Press, Bocca Raton, Fla.).

In a specific embodiment of the present invention, where the Therapeutic is a nucleic acid encoding a protein, the Therapeutic nucleic acid may be administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus (See, e.g., Joliot, et al., 1991. Proc Natl Acad Sci USA 88:1864-1868), and the like. Alternatively, a nucleic acid Therapeutic can be introduced intracellularly and incorporated within host cell DNA for expression, by homologous recombination.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of the Therapeutic of the invention which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. However, suitable dosage ranges for intravenous administration of the Therapeutics of the present invention are generally about 20-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Polynucleotides of the present invention can also be used for gene therapy. Gene therapy refers to therapy that is performed by the administration of a specific nucleic acid to a subject. Delivery of the Therapeutic nucleic acid into a mammalian subject may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. Polynucleotides of the invention may also be administered by other known methods for introduction of nucleic acid into a cell or organism (including, without limitation, in the form of viral vectors or naked DNA). Any of the methodologies relating to gene therapy available within the art may be used in the practice of the present invention. See e.g., Goldspiel, et al., 1993. Clin Pharm 12:488-505.

Cells may also be cultured ex vivo in the presence of therapeutic agents or proteins of the present invention in order to proliferate or to produce a desired effect on or activity in such cells. Treated cells can then be introduced in vivo for therapeutic purposes.

Assessing Efficacy of an Obesity Treatment in a Subject

The differentially expressed OB sequences identified herein also allow for the course of treatment of a pathophysiology to be monitored. In this method, a test cell population is provided from a subject undergoing treatment for obesity. If desired, test cell populations can be taken from the subject at various time points before, during, or after treatment. Expression of one or more of the OB sequences, e.g., OBs:1-6, in the cell population is then measured and compared to a reference cell population which includes cells whose-pathophysiologic state is known. Preferably, the reference cells have not been exposed to the treatment.

If the reference cell population contains cells not exposed to the treatment and not suffering from the disorder, then a difference in expression between OB sequences in the test population and this reference cell population indicates the treatment is not efficacious. However, a similarity in expression between OB sequences in the test cell population and the reference cell population described above indicates that the treatment is efficacious.

By “efficacious” is meant that the treatment leads to a decrease in the pathophysiology in a subject. When treatment is applied prophylactically, “efficacious” means that the treatment retards or prevents a pathophysiology. For example, if the obesity treatment is “efficacious”, it decreases the degree of obesity in a subject.

Efficacy can be determined in association with any known method for treating the particular pathophysiology.

Identifying Agents that Modulate Obesity

Also included in the invention are methods of identifying agents that modulate obesity. One method includes contacting one or more OB polypeptides with a test agent and detecting a complex between the test agent and the polypeptide. A presence of a complex indicates that the test agent modulates obesity. Absence of a complex indicates that the test agent does not modulate obesity.

By “modulate obesity” is meant that the test agent either increases or decreases the expression of one or more of the OB nucleic acid sequences.

A test agent can be, e.g. antibodies to the polypeptides encoded by OBs:1-6, peptides, peptidomimetics, small molecules or other drugs.

The test agent may be a known or an unknown therapeutic agent. Examples of know therapeutic agents include, but are not limited to, dexfenfluramine, sibutramine, beta3-adrenergic agonists, and olistat.

Methods of Modulating the Activity of OB Proteins

The invention provides a method for identifying modulators, i.e., candidate or test compounds or agents (e.g., antibodies to the polypeptides encoded by OBs:1-6, peptides, peptidomimetics, small molecules or other drugs) that bind to OB proteins or have a stimulatory or inhibitory effect on, for example, OB expression or OB activity.

In one embodiment, the invention provides assays for screening candidate or test compounds which bind to or modulate the activity of the membrane-bound form of an OB protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des 12:145).

Examples of methods for the synthesis of molecular libraries can be found in the art, for example in: DeWitt et al. (1993) Proc Natl Acad Sci U.S.A. 90:6909; Erb et al. (1994) Proc Natl Acad Sci U.S.A. 91:11422; Zuckermann et al. (1994) J Med Chem 37:2678; Cho et al. (1993) Science 261:1303; Carrell et al. (1994) Angew Chem Int Ed Engl 33:2059; Carell et al. (1994) Angew Chem Int Ed Engl 33:2061; and Gallop et al. (1994) J Med Chem 37:1233.

Libraries of compounds may be presented in solution (e.g., Houghten (1992) Biotechniques 13:412-421), or on beads (Lam (1991) Nature 354:82-84), on chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc Natl Acad Sci U.S.A. 87:6378-6382; Felici (1991) J Mol Biol 222:301-310; Ladner above.).

In one embodiment, an assay is a cell-based assay in which a cell which expresses a membrane-bound form of OB protein, or a biologically active portion thereof, on the cell surface is contacted with a test compound and the ability of the test compound to bind to an OB protein determined. The cell, for example, can be of mammalian origin or a yeast cell. Determining the ability of the test compound to bind to the OB protein can be accomplished, for example, by coupling the test compound with a radioisotope or enzymatic label such that binding of the test compound to the OB protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of OB protein, or a biologically active portion thereof, on the cell surface with a known compound which binds OB to form an assay mixture, contacting the assay mixture with a test compound, and determining the ability of the test compound to interact with an OB protein, wherein determining the ability of the test compound to interact with an OB protein comprises determining the ability of the test compound to preferentially bind to OB or a biologically active portion thereof as compared to the known compound.

In another embodiment, an assay is a cell-based assay comprising contacting a cell expressing a membrane-bound form of OB protein, or a biologically active portion thereof, on the cell surface with a test compound and determining the ability of the test compound to modulate (e.g., stimulate or inhibit) the activity of the OB protein or biologically active portion thereof. Determining the ability of the test compound to modulate the activity of OB or a biologically active portion thereof can be accomplished, for example, by determining the ability of the OB protein to bind to or interact with an OB target molecule. As used herein, a “target molecule” is a molecule with which an OB protein binds or interacts in nature, for example, a molecule on the surface of a cell which expresses an OB interacting protein, a molecule on the surface of a second cell, a molecule in the extracellular milieu, a molecule associated with the internal surface of a cell membrane or a cytoplasmic molecule. An OB target molecule can be a non-OB molecule or an OB protein or polypeptide of the present invention. In one embodiment, an OB target molecule is a component of a signal transduction pathway that facilitates transduction of an extracellular signal (e.g. a signal generated by binding of a compound to a membrane-bound OB molecule) through the cell membrane and into the cell. The target, for example, can be a second intercellular protein that has catalytic activity or a protein that facilitates the association of downstream signaling molecules with OB.

Determining the ability of the OB protein to bind to or interact with an OB target molecule can be accomplished by one of the methods described above for determining direct binding. In one embodiment, determining the ability of the OB protein to bind to or interact with an OB target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an OB-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the present invention is a cell-free assay comprising contacting an OB protein or biologically active portion thereof with a test compound and determining the ability of the test compound to bind to the OB protein or biologically active portion thereof. Binding of the test compound to the OB protein can be determined either directly or indirectly as described above. In one embodiment, the assay comprises contacting the OB protein or biologically active portion thereof with a known compound which binds OB to form an assay mixture, contacting the assay mixture with a test compound, and determining the ability of the test compound to interact with an OB protein, wherein determining the ability of the test compound to interact with an OB protein comprises determining the ability of the test compound to preferentially bind to OB or biologically active portion thereof as compared to the known compound.

In another embodiment, an assay is a cell-free assay comprising contacting OB protein or biologically active portion thereof with a test compound and determining the ability of the test compound to modulate (e.g. stimulate or inhibit) the activity of the OB protein or biologically active portion thereof. Determining the ability of the test compound to modulate the activity of OB can be accomplished, for example, by determining the ability of the OB protein to bind to an OB target molecule by one of the methods described above for determining direct binding. In an alternative embodiment, determining the ability of the test compound to modulate the activity of OB can be accomplished by determining the ability of the OB protein further modulate an OB target molecule. For example, the catalytic/enzymatic activity of the target molecule on an appropriate substrate can be determined as previously described.

In yet another embodiment, the cell-free assay comprises contacting the OB protein or biologically active portion thereof with a known compound which binds OB to form an assay mixture, contacting the assay mixture with a test compound, and determining the ability of the test compound to interact with an OB protein, wherein determining the ability of the test compound to interact with an OB protein comprises determining the ability of the OB protein to preferentially bind to or modulate the activity of an OB target molecule.

The cell-free assays of the present invention are amenable to use of both the soluble form or the membrane-bound form of OB. In the case of cell-free assays comprising the membrane-bound form of OB, it may be desirable to utilize a solubilizing agent such that the membrane-bound form of OB is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114. Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the present invention, it may be desirable to immobilize either OB or its target molecule to facilitate separation of complexed from uncomplexed forms of one or both of the proteins, as well as to accommodate automation of the assay. Binding of a test compound to OB, or interaction of OB with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-OB fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or OB protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of OB binding or activity determined using standard techniques.

Other techniques for immobilizing proteins on matrices can also be used in the screening assays of the invention. For example, either OB or its target molecule can be immobilized utilizing conjugation of biotin and streptavidin. Biotinylated OB or target molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with OB or target molecules, but which do not interfere with binding of the OB protein to its target molecule, can be derivatized to the wells of the plate, and unbound target or OB trapped in the wells by antibody conjugation. Methods for detecting such complexes, in addition to those described above for the GST-immobilized complexes, include immunodetection of complexes using antibodies reactive with the OB or target molecule, as well as enzyme-linked assays that rely on detecting an enzymatic activity associated with the OB or target molecule.

In another embodiment, modulators of OB expression are identified in a method wherein a cell is contacted with a candidate compound and the expression of OB mRNA or protein in the cell is determined. The level of expression of OB mRNA or protein in the presence of the candidate compound is compared to the level of expression of OB mRNA or protein in the absence of the candidate compound. The candidate compound can then be identified as a modulator of OB expression based on this comparison. For example, when expression of OB mRNA or protein is greater (statistically significantly greater) in the presence of the candidate compound than in its absence, the candidate compound is identified as a stimulator of OB mRNA or protein expression. Alternatively, when expression of OB mRNA or protein is less (statistically significantly less) in the presence of the candidate compound than in its absence, the candidate compound is identified as an inhibitor of OB mRNA or protein expression. The level of OB mRNA or protein expression in the cells can be determined by methods described herein for detecting OB mRNA or protein.

In yet another aspect of the invention, the OB proteins can be used as “bait proteins” in a two-hybrid assay or three hybrid assay (see, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; Iwabuchi et al. (1993) Oncogene 8:1693-1696; and Brent WO94/10300), to identify other proteins that bind to or interact with OB (“OB-binding proteins” or “OB-bp”) and modulate OB activity. Such OB-binding proteins are also likely to be involved in the propagation of signals by the OB proteins as, for example, upstream or downstream elements of the OB pathway.

The two-hybrid system is based on the modular nature of most transcription factors, which consist of separable DNA-binding and activation domains. Briefly, the assay utilizes two different DNA constructs. In one construct, the gene that codes for OB is fused to a gene encoding the DNA binding domain of a known transcription factor (e.g., GALA). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an OB-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein, which interacts with OB.

This invention further pertains to novel agents identified by the above-described screening assays and uses thereof for treatments as described herein.

Methods of Detecting OB Proteins

The invention also provides a method for detecting the presence or absence of OB in a biological sample. The method includes obtaining a biological sample from a test subject and contacting the biological sample with a compound or an agent capable of detecting OB protein or nucleic acid (e.g., mRNA, genomic DNA) that encodes OB protein such that the presence of OB is detected in the biological sample. An agent for detecting OB mRNA or genomic DNA is a labeled nucleic acid probe capable of hybridizing to OB mRNA or genomic DNA. The nucleic acid probe can be, for example, a full-length OB nucleic acid, such as the nucleic acid of SEQ ID NO: 1, or a portion thereof, such as an oligonucleotide of at least 15, 30, 50, 100, 250 or 500 nucleotides in length and sufficient to specifically hybridize under stringent conditions to OB mRNA or genomic DNA. Other suitable probes for use in the diagnostic assays of the invention are described herein.

An agent for detecting OB protein is an antibody capable of binding to OB protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect OB mRNA, protein, or genomic DNA in a biological sample in vitro as well as in vivo. For example, in vitro techniques for detection of OB mRNA include Northern hybridizations and in situ hybridizations. In vitro techniques for detection of OB protein include enzyme linked immunosorbent assays (ELISAs), Western blots, immunoprecipitations and immunofluorescence. In vitro techniques for detection of OB genomic DNA include Southern hybridizations. Furthermore, in vivo techniques for detection of OB protein include introducing into a subject a labeled anti-OB antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting OB protein, mRNA, or genomic DNA, such that the presence of OB protein, mRNA or genomic DNA is detected in the biological sample, and comparing the presence of OB protein, mRNA or genomic DNA in the control sample with the presence of OB protein, mRNA or genomic DNA in the test sample.

OB Nucleic Acids

Also provided in the invention is a novel nucleic acid that includes a nucleic acid sequence selected from the group consisting of OB6, or its complement, as well as vectors and cells including these nucleic acids. Also provided are polypeptides encoded by OB nucleic acid or biologically active portions thereof.

Also included in the invention are nucleic acid fragments sufficient for use as hybridization probes to identify OB-encoding nucleic acids (e.g., OB mRNA) and fragments for use as polymerase chain reaction (PCR) primers for the amplification or mutation of OB nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt) or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules, which are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated OB nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of OB6, or its complement, can be isolated using standard molecular biology techniques and the sequence information provided herein. Using all or a portion of these nucleic acid sequences as a hybridization probe, OB nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.).

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to OB nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having at least about 10 nt and as many as 50 nt, preferably about 15 nt to 30 nt. They may be chemically synthesized and may be used as probes.

In another embodiment, an isolated nucleic acid molecule of the invention comprises a nucleic acid molecule that is a complement of the nucleotide sequence of OB6. In another embodiment, an isolated nucleic acid molecule of the invention comprises a nucleic acid molecule that is a complement of the nucleotide sequence shown in any of these sequences, or a portion of any of these nucleotide sequences. A nucleic acid molecule that is complementary to the nucleotide sequence of OB6 is one that is sufficiently complementary to the nucleotide sequence shown, such that it can hydrogen bond with little or no mismatches to the nucleotide sequences shown, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of OB6, e.g., a fragment that can be used as a probe or primer or a fragment encoding a biologically active portion of OB6. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention-include, but are not limited to, molecules comprising regions that are substantially homologous to the nucleic acids or proteins of the invention, in various embodiments, by at least about 45%, 50%, 70%, 80%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENTPROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which in incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of an OB polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for an OB polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding a human OB protein. Homologous nucleic acid sequences include those nucleic acid sequences that encode conservative amino acid substitutions (see below) in an OB polypeptide, as well as a polypeptide having an OB activity. A homologous amino acid sequence does not encode the amino acid sequence of a human OB polypeptide.

The nucleotide sequence determined from the cloning of human OB genes allows for the generation of probes and primers designed for use in identifying and/or cloning OB homologues in other cell types, e.g., from other tissues, as well as OB homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of a nucleic acid comprising an OB sequence, or an anti-sense strand nucleotide sequence of a nucleic acid comprising an OB sequence, or of a naturally occurring mutant of these sequences.

Probes based on human OB nucleotide sequences can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress an OB protein, such as by measuring a level of an OB-encoding nucleic acid in a sample of cells from a subject e.g., detecting OB mRNA levels or determining whether a genomic OB gene has been mutated or deleted.

“A polypeptide having a biologically active portion of OB” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of OB” can be prepared by isolating a portion of OB6, that encodes a polypeptide having an OB biological activity, expressing the encoded portion of OB protein (e.g., by recombinant expression in vitro) and assessing the activity of the encoded portion of OB. For example, a nucleic acid fragment encoding a biologically active portion of an OB polypeptide can optionally include an ATP-binding domain.

In another embodiment, a nucleic acid fragment encoding a biologically active portion of OB includes one or more regions.

The OB nucleic acid sequences according to the invention can be used to treat obesity in a subject. The subject is preferably a mammal, more preferably a human. In one aspect, the expression status of such a nucleic acid sequence used to treat obesity in a subject is regulated by leptin. By “expression status” is meant the degree to which the nucleic acid sequence is expressed. In specific embodiments, the expression status of the nucleic acid used to treat obesity in a subject can either be up-regulated (i.e. OBs:3) or down-regulated (i.e. OBs:1,2, and 4-6). Additionally, in another aspect, the nucleic acid sequence used to treat obesity in a subject is expressed in the pituitary gland.

OB Variants

The invention further encompasses nucleic acid molecules that differ from the disclosed or referenced OB nucleotide sequences due to degeneracy of the genetic code. These nucleic acids thus encode the same OB protein as that encoded by nucleotide sequence comprising an OB nucleic acid as shown in, e.g., OB6.

In addition to the OB nucleotide sequence shown in OB6, it will be appreciated by those skilled in the art that DNA sequence polymorphisms that lead to changes in the amino acid sequences of an OB polypeptide may exist within a population (e.g., the human population). Such genetic polymorphism in the OB gene may exist among individuals within a population due to natural allelic variation. As used herein, the terms “gene” and “recombinant gene” refer to nucleic acid molecules comprising an open reading frame encoding an OB protein, preferably a mammalian OB protein. Such natural allelic variations can typically result in 1-5% variance in the nucleotide sequence of the OB gene. Any and all such nucleotide variations and resulting amino acid polymorphisms in OB that are the result of natural allelic variation and that do not alter the functional activity of OB are intended to be within the scope of the invention.

Moreover, nucleic acid molecules encoding OB proteins from other species, and thus that have a nucleotide sequence that differs from the human sequence of OB6, are intended to be within the scope of the invention. Nucleic acid molecules corresponding to natural allelic variants and homologues of the OB DNAs of the invention can be isolated based on their homology to the human OB nucleic acids disclosed herein using the human cDNAs, or a portion thereof, as a hybridization probe according to standard hybridization techniques under stringent hybridization conditions. For example, a soluble human OB DNA can be isolated based on its homology to human membrane-bound OB. Likewise, a membrane-bound human OB DNA can be isolated based on its homology to soluble human OB.

Accordingly, in another embodiment, an isolated nucleic acid molecule of the invention is at least 6 nucleotides in length and hybridizes under stringent conditions to the nucleic acid molecule comprising the nucleotide sequence of OB6. In another embodiment, the nucleic acid is at least 10, 25, 50, 100, 250 or 500 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding OB proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of OB6 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of OB6 or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of OB6 or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL. Stockton Press, NY; Shilo et al., 1981, Proc Natl Acad Sci USA 78: 6789-6792.

Conservative Mutations

In addition to naturally-occurring allelic variants of the OB sequence that may exist in the population, the skilled artisan will further appreciate that changes can be introduced into an OB nucleic acid or directly into an OB polypeptide sequence without altering the functional ability of the OB protein. In some embodiments, the nucleotide sequence of OB6 will be altered, thereby leading to changes in the amino acid sequence of the encoded OB protein. For example, nucleotide substitutions that result in amino acid substitutions at various “non-essential” amino acid residues can be made in the sequence of OB6. A “non-essential” amino acid residue is a residue that can be altered from the wild-type sequence of OB without altering the biological activity, whereas an “essential” amino acid residue is required for biological activity. For example, amino acid residues that are conserved among the OB proteins of the present invention, are predicted to be particularly unamenable to alteration.

In addition, amino acid residues that are conserved among family members of the OB proteins of the present invention, are also predicted to be particularly unamenable to alteration. As such, these conserved domains are not likely to be amenable to mutation. Other amino acid residues, however, (e.g., those that are not conserved or only semi-conserved among members of the OB proteins) may not be essential for activity and thus are likely to be amenable to alteration.

Another aspect of the invention pertains to nucleic acid molecules encoding OB proteins that contain changes in amino acid residues that are not essential for activity. Such OB proteins differ in amino acid sequence from the amino acid sequences of polypeptides encoded by nucleic acids containing OB6, yet retain biological activity. In one embodiment, the isolated nucleic acid molecule comprises a nucleotide sequence encoding a protein, wherein the protein comprises an amino acid sequence at least about 45% homologous, more preferably 60%, and still more preferably at least about 70%, 80%, 90%, 95%, 98%; and most preferably at least about 99% homologous to the amino acid sequence of the amino acid sequences of polypeptides encoded by nucleic acids comprising OB6.

An isolated nucleic acid molecule encoding an OB protein homologous to can be created by introducing one or more nucleotide substitutions, additions or deletions into the nucleotide sequence of a nucleic acid comprising OB6, such that one or more amino acid substitutions, additions or deletions are introduced into the encoded protein.

Mutations can be introduced into a nucleic acid comprising OB6 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in OB is replaced with another amino acid residue from the same side chain family. Alternatively, in another embodiment, mutations can be introduced randomly along all or part of an OB coding sequence, such as by saturation mutagenesis, and the resultant mutants can be screened for OB biological activity to identify mutants that retain activity. Following mutagenesis of the nucleic acid, the encoded protein can be expressed by any recombinant technology known in the art and the activity of the protein can be determined.

In one embodiment, a mutant OB protein can be assayed for (1) the ability to form protein:protein interactions with other OB proteins, other cell-surface proteins, or biologically active portions thereof, (2) complex formation between a mutant OB protein and an OB ligand; (3) the ability of a mutant OB protein to bind to an intracellular target protein or biologically active portion thereof; (e.g., avidin proteins); (4) the ability to bind ATP; or (5) the ability to specifically bind an OB protein antibody.

In other embodiment, the fragment of the complementary polynucleotide sequence of OB6, wherein the fragment of the complementary polynucleotide sequence hybridizes to the first sequence.

In other specific embodiments, the nucleic acid is RNA or DNA. The fragment or the fragment of the complementary polynucleotide sequence of OB6, wherein the fragment is between about 10 and about 100 nucleotides in length, e.g., between about 10 and about 90 nucleotides in length, or about 10 and about 75 nucleotides in length, about 10 and about 50 bases in length, about 10 and about 40 bases in length, or about 15 and about 30 bases in length.

Antisense

Another aspect of the invention pertains to isolated antisense nucleic acid molecules that are hybridizable to or complementary to the nucleic acid molecule comprising the nucleotide sequence of an OB sequence or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire OB coding strand, or to only a portion thereof. Nucleic acid molecules encoding fragments, homologs, derivatives and analogs of an OB protein, or antisense nucleic acids complementary to a nucleic acid comprising an OB nucleic acid sequence are additionally provided.

In one embodiment, an antisense nucleic acid molecule is antisense to a “coding region” of the coding strand of a nucleotide sequence encoding OB. The term “coding region” refers to the region of the nucleotide sequence comprising codons, which are translated into amino acid residues. In another embodiment, the antisense nucleic acid molecule is antisense to a “noncoding region” of the coding strand of a nucleotide sequence encoding OB. The term “noncoding region” refers to 5′ and 3′ sequences which flank the coding region that are not translated into amino acids (i.e., also referred to as 5′ and 3′ untranslated regions).

Given the coding strand sequences encoding OB disclosed herein, antisense nucleic acids of the invention can be designed according to the rules of Watson and Crick or Hoogsteen base pairing. The antisense nucleic acid molecule can be complementary to the entire coding region of OB mRNA, but more preferably is an oligonucleotide that is antisense to only a portion of the coding or noncoding region of OB mRNA. For example, the antisense oligonucleotide can be complementary to the region surrounding the translation start site of OB mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2.2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an OB protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave OB mRNA transcripts to thereby inhibit translation of OB mRNA. A ribozyme having specificity for an OB-encoding nucleic acid can be designed based upon the nucleotide sequence of an OB DNA disclosed herein. For example, a derivative of a Tetrahymena L-19 IVS RNA can be constructed in which the nucleotide sequence of the active site is complementary to the nucleotide sequence to be cleaved in an OB-encoding mRNA. See, e.g., Cech et al. U.S. Pat. No. 4,987,071; and Cech et al. U.S. Pat. No. 5,116,742. Alternatively, OB mRNA can be used to select a catalytic RNA having a specific ribonuclease activity from a pool of RNA molecules. See, e.g., Bartel et al., (1993) Science 261:1411-1418.

Alternatively, OB gene expression can be inhibited by targeting nucleotide sequences complementary to the regulatory region of an OB nucleic acid (e.g., the OB promoter and/or enhancers) to form triple helical structures that prevent transcription of the OB gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of OB can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of OB can be used in therapeutic and diagnostic applications. For example, PNAs can be used as antisense or antigene agents for sequence-specific modulation of gene expression by, e.g., inducing transcription or translation arrest or inhibiting replication. PNAs of OB can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of OB can be modified, e.g., to enhance their stability or cellular uptake, by attaching lipophilic or other helper groups to PNA, by the formation of —PNA-DNA chimeras, or by the use of liposomes or other techniques of drug delivery known in the art. For example, PNA-DNA chimeras of OB can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

OB Polypeptides

One aspect of the invention pertains to isolated OB proteins, and biologically active portions thereof, or derivatives, fragments, analogs or homologs thereof. Also provided are polypeptide fragments suitable for use as immunogens to raise anti-OB antibodies. In one embodiment, native OB proteins can be isolated from cells or tissue sources by an appropriate purification scheme using standard protein purification techniques. In another embodiment, OB proteins are produced by recombinant DNA techniques. Alternative to recombinant expression, an OB protein or polypeptide can be synthesized chemically using standard peptide synthesis techniques.

An “isolated” or “purified” protein or biologically active portion thereof is substantially free of cellular material or other contaminating proteins from the cell or tissue source from which the OB protein is derived, or substantially free from chemical precursors or other chemicals when chemically synthesized. The language “substantially free of cellular material” includes preparations of OB protein in which the protein is separated from cellular components of the cells from which it is isolated or recombinantly produced. In one embodiment, the language “substantially free of cellular material” includes preparations of OB protein having less than about 30% (by dry weight) of non-OB protein (also referred to herein as a “contaminating protein”), more preferably less than about 20% of non-OB protein, still more preferably less than about 10% of non-OB protein, and most preferably less than about 5% non-OB protein. When the OB protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of OB protein in which the protein is separated from chemical precursors or other chemicals that are involved in the synthesis of the protein. In one embodiment, the language “substantially free of chemical precursors or other chemicals” includes preparations of OB protein having less than about 30% (by dry weight) of chemical precursors or non-OB chemicals, more preferably less than about 20% chemical precursors or non-OB chemicals, still more preferably less than about 10% chemical precursors or non-OB chemicals, and most preferably less than about 5% chemical precursors or non-OB chemicals.

Biologically active portions of an OB protein include peptides comprising amino acid sequences sufficiently homologous to or derived from the amino acid sequence of the OB protein, e.g., the amino acid sequence encoded by a nucleic acid comprising OB6 that include fewer amino acids than the full length OB proteins, and exhibit at least one activity of an OB protein. Typically, biologically active portions comprise a domain or motif with at least one activity of the OB protein. A biologically active portion of an OB protein can be a polypeptide, which is, for example, 10, 25, 50, 100 or more amino acids in length.

A biologically active portion of an OB protein of the present invention may contain at least one of the above-identified domains conserved between the OB proteins. An alternative biologically active portion of an OB protein may contain at least two of the above-identified domains. Another biologically active portion of an OB protein may contain at least three of the above-identified domains. Yet another biologically active portion of an OB protein of the present invention may contain at least four of the above-identified domains.

Moreover, other biologically active portions, in which other regions of the protein are deleted, can be prepared by recombinant techniques and evaluated for one or more of the functional activities of a native OB protein.

In some embodiments, the OB protein is substantially homologous to one of these OB proteins and retains its the functional activity, yet differs in amino acid sequence due to natural allelic variation or mutagenesis, as described in detail below.

In specific embodiments, the invention includes an isolated polypeptide comprising an amino acid sequence that is 80% or more identical to the sequence of a polypeptide whose expression is modulated in a mammal.

The OB polypeptides according to the invention can be used to treat obesity in a subject. The subject is preferably a mammal, more preferably a human. In one aspect, the expression status of such a polypeptide used to treat obesity in a subject is regulated by leptin. By “expression status” is meant the degree to which the polypeptide is expressed. In specific embodiments, the expression status of the polypeptides used to treat obesity in a subject can either be up-regulated (i.e. OBs:3) or down-regulated (i.e. OBs:1,2, and 4-6). Additionally, in another aspect, the polypeptide used to treat obesity in a subject is secreted from the pituitary gland.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of a DNA sequence comprising OB6.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

OB Chimeric and Fusion Proteins

The invention also provides OB chimeric or fusion proteins. As used herein, an OB “chimeric protein” or “fusion protein” comprises an OB polypeptide operatively linked to a non-OB polypeptide. A “OB polypeptide” refers to a polypeptide having an amino acid sequence corresponding to OB, whereas a “non-OB polypeptide” refers to a polypeptide having an amino acid sequence corresponding to a protein that is not substantially homologous to the OB protein, e.g., a protein that is different from the OB protein and that is derived from the same or a different organism. Within an OB fusion protein the OB polypeptide can correspond to all or a portion of an OB protein. In one embodiment, an OB fusion protein comprises at least one biologically active portion of an OB protein. In another embodiment, an OB fusion protein comprises at least two biologically active portions of an OB protein. In yet another embodiment, an OB fusion protein comprises at least three biologically active portions of an OB protein. Within the fusion protein, the term “operatively linked” is intended to indicate that the OB polypeptide and the non-OB polypeptide are fused in-frame to each other. The non-OB polypeptide can be fused to the N-terminus or C-terminus of the OB polypeptide.

For example, in one embodiment an OB fusion protein comprises an OB domain operably linked to the extracellular domain of a second protein. Such fusion proteins can be further utilized in screening assays for compounds which modulate OB activity (such assays are described in detail below).

In yet another embodiment, the fusion protein is a GST-OB fusion protein in which the OB sequences are fused to the C-terminus of the GST (i.e., glutathione S-transferase) sequences. Such fusion proteins can facilitate the purification of recombinant OB.

In another embodiment, the fusion protein is an OB protein containing a heterologous signal sequence at its N-terminus. For example, a native OB signal sequence can be removed and replaced with a signal sequence from another protein. In certain host cells (e.g., mammalian host cells), expression and/or secretion of OB can be increased through use of a heterologous signal sequence.

In yet another embodiment, the fusion protein is an OB-immunoglobulin fusion protein in which the OB sequences comprising one or more domains are fused to sequences derived from a member of the immunoglobulin protein family. The OB-immunoglobulin fusion proteins of the invention can be incorporated into pharmaceutical compositions and administered to a subject to inhibit an interaction between an OB ligand and an OB protein on the surface of a cell, to thereby suppress OB-mediated signal transduction in vivo. The OB-immunoglobulin fusion proteins can be used to affect the bioavailability of an OB cognate ligand. Inhibition of the OB ligand/OB interaction may be useful therapeutically for both the treatments of proliferative and differentiative disorders, as well as modulating (e.g. promoting or inhibiting) cell survival. Moreover, the OB-immunoglobulin fusion proteins of the invention can be used as immunogens to produce anti-OB antibodies in a subject, to purify OB ligands, and in screening assays to identify molecules that inhibit the interaction of OB with an OB ligand.

An OB chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An OB-encoding nucleic acid can be cloned into such an expression vector such that the fusion moiety is linked in-frame to the OB protein.

OB Agonists and Antagonists

The present invention also pertains to variants of the OB proteins that function as either OB agonists (mimetics) or as OB antagonists. Variants of the OB protein can be generated by mutagenesis, e.g., discrete point mutation or truncation of the OB protein. An agonist of the OB protein can retain substantially the same, or a subset of, the biological activities of the naturally occurring form of the OB protein. An antagonist of the OB protein can inhibit one or more of the activities of the naturally occurring form of the OB protein by, for example, competitively binding to a downstream or upstream member of a cellular signaling cascade which includes the OB protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the OB proteins.

Variants of the OB protein that function as either OB agonists (mimetics) or as OB antagonists can be identified by screening combinatorial libraries of mutants, e.g., truncation mutants, of the OB protein for OB protein agonist or antagonist activity. In one embodiment, a variegated library of OB variants is generated by combinatorial mutagenesis at the nucleic acid level and is encoded by a variegated gene library. A variegated library of OB variants can be produced by, for example, enzymatically ligating a mixture of synthetic oligonucleotides into gene sequences such that a degenerate set of potential OB sequences is expressible as individual polypeptides, or alternatively, as a set of larger fusion proteins (e.g., for phage display) containing the set of OB sequences therein. There are a variety of methods, which can be used to produce libraries of potential OB variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential OB sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the OB protein coding sequence can be used to generate a variegated population of OB fragments for screening and subsequent selection of variants of an OB protein. In one embodiment, a library of coding sequence fragments can be generated by treating a double stranded PCR fragment of an OB coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the OB protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of OB proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify OB variants (Arkin and Yourvan (1992) PNAS 89:7811-7815; Delgrave et al. (1993) Protein Engineering 6:327-331).

Anti-OB Antibodies

An isolated OB protein, or a portion or fragment thereof, can be used as an immunogen to generate antibodies that bind OB using standard techniques for polyclonal and monoclonal antibody preparation. The full-length OB protein can be used or, alternatively, the invention provides antigenic peptide fragments of OB for use as immunogens. The antigenic peptide of OB comprises at least 8 amino acid residues of the amino acid sequence encoded by a nucleic acid comprising the nucleic acid sequence shown in OB6 and encompasses an epitope of OB such that an antibody raised against the peptide forms a specific immune complex with OB. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of OB that are located on the surface of the protein, e.g., hydrophilic regions. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety.

OB polypeptides or derivatives, fragments, analogs or homologs thereof, may be utilized as immunogens in the generation of antibodies that immunospecifically-bind these protein components. The term “antibody” as used herein refers to immunoglobulin molecules and immunologically active portions of immunoglobulin molecules, i.e., molecules that contain an antigen binding site that specifically binds (immunoreacts with) an antigen. Such antibodies include, but are not limited to, polyclonal, monoclonal, chimeric, single chain, F_{ab} and F_{(ab)2} fragments, and an F_{ab} expression library. Various procedures known within the art may be used for the production of polyclonal or monoclonal antibodies to an OB protein sequence, or derivatives, fragments, analogs or homologs thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed OB protein or a chemically synthesized OB polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against OB can be isolated from the mammal (e.g., from the blood) and further purified by well-known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of OB. A monoclonal antibody composition thus typically displays a single binding affinity for a particular OB protein with which it immunoreacts. For preparation of monoclonal antibodies directed towards a particular OB protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

According to the invention, techniques can be adapted for the production of single-chain antibodies specific to an OB protein (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for an OB protein or derivatives, fragments, analogs or homologs thereof. Non-human antibodies can be “humanized” by techniques well known in the art. See e.g., U.S. Pat. No. 5,225,539. Antibody fragments that contain the idiotypes to an OB protein may be produced by techniques known in the art including, but not limited to: (i) an F_{(ab)2} fragment produced by pepsin digestion of an antibody molecule; (ii) an F_{ab} fragment generated by reducing the disulfide bridges of an F_{(ab)2} fragment; (iii) an F_{ab} fragment generated by the treatment of the antibody molecule with papain and a reducing agent and (iv) Fᵥ fragments.

Additionally, recombinant anti-OB antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988) J Natl Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J Immunol 141:4053-4060.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an OB protein is facilitated by generation of hybridomas that bind to the fragment of an OB protein possessing such a domain. Antibodies that are specific for one or more domains within an OB protein, e.g., domains spanning the above-identified conserved regions of OB family proteins, or derivatives, fragments, analogs or homologs thereof, are also provided herein.

Anti-OB antibodies may be used in methods known within the art relating to the localization and/or quantitation of an OB protein (e.g., for use in measuring levels of the OB protein within appropriate physiological samples, for use in diagnostic methods, for use in imaging the protein, and the like). In a given embodiment, antibodies for OB proteins, or derivatives, fragments, analogs or homologs thereof, that contain the antibody derived binding domain, are utilized as pharmacologically-active compounds [hereinafter “Therapeutics”].

An anti-OB antibody (e.g., monoclonal antibody) can be used to isolate OB by standard techniques, such as affinity chromatography or immunoprecipitation. An anti-OB antibody can facilitate the purification of natural OB from cells and of recombinantly produced OB expressed in host cells. Moreover, an anti-OB antibody can be used to detect OB protein (e.g., in a cellular lysate or cell supernatant) in order to evaluate the abundance and pattern of expression of the OB protein. Anti-OB antibodies can be used diagnostically to monitor protein levels in tissue as part of a clinical testing procedure, e.g., to, for example, determine the efficacy of a given treatment regimen. Detection can be facilitated by coupling (i e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

OB Recombinant Expression Vectors and Host Cells

Another aspect of the invention pertains to vectors, preferably expression vectors, containing a nucleic acid encoding OB protein, or derivatives, fragments, analogs or homologs thereof. As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., OB proteins, mutant forms of OB, fusion proteins, etc.).

The recombinant expression vectors of the invention can be designed for expression of OB in prokaryotic or eukaryotic cells. For example, OB can be expressed in bacterial cells such as E. Coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the OB expression vector is a yeast expression vector. Examples of vectors for expression in yeast S. cerevisiae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, OB can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to OB mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, OB protein can be expressed in bacterial cells such as E. Coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding OB or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an OB protein. Accordingly, the invention further provides methods for producing OB protein using the host cells of the invention. In one embodiment, the method comprises culturing the host cell of invention (into which a recombinant expression vector encoding OB has been introduced) in a suitable medium such that OB protein is produced. In another embodiment, the method further comprises isolating OB from the medium or the host cell.

Kits and Nucleic Acid Collections for Identifying OB Nucleic Acids

In another aspect, the invention provides a kit useful for examining a pathophysiology associated with obesity. The kit can include nucleic acids that detect two or more OB sequences. In preferred embodiments, the kit includes reagents which detect 3, 4, 5, or all of the OB nucleic acid sequences.

The kit or plurality may include, e.g., sequence homologous to OB nucleic acid sequences, or sequences which, can specifically identify one or more OB nucleic acid sequences.

The invention provides for a kit comprising one or more reagents for detecting two or more nucleic acid sequences selected from the group consisting of OBs:1-6. In various embodiments, the expression of 2, 3, 4, 5, or more of the sequences represented by OBs:1-6 are measured. The kit can identify the enumerated nucleic acids by, e.g., having homologous nucleic acid sequences, such as oligonucleotide sequences, complementary to a portion of the recited nucleic acids, or antibodies to proteins encoded by the genes.

The invention also includes an array of probe nucleic acids. These probe nucleic acid sequences detect two or more nucleic acid sequences selected from the group consisting of OBs:1-6. In various embodiments, the expression of 2, 3, 4, 5, or more of the sequences represented by OBs:1-6 are identified.

The probe nucleic acids in the array can detect the enumerated nucleic acids by, e.g., having homologous nucleic acid sequences, such as oligonucleotide sequences, complementary to a portion of the recited nucleic acids. The substrate array can be on, e.g., a solid substrate, e.g., a “chip”, as described in U.S. Pat. No. 5,744,305.

The invention also includes an isolated plurality of nucleic acid sequences. The plurality typically includes two or more of the nucleic acid sequences represented by OBs:1-6. In various embodiments, the plurality includes 2, 3, 4, 5, or more of the sequences represented by OBs:1-6.

An “isolated” nucleic acid molecule is one that is separated from other nucleic acid molecules, which are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, and “isolated” nucleic acid is free of sequences which naturally flank the nucleic acid (i.e., sequences located at the 5′ and 3′ ends of the nucleic acid) in the genomic DNA of the organism from which the nucleic acid is derived. In various embodiments, the isolated nucleic acid molecule can contain less than about 50 kb, 25 kb, 5 kb, 4 kb, 3 kb, 2 kb, 1 kb, 0.5 kb, or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or cultural medium when produced by recombinant techniques, or of chemical precursors or other chemicals when chemically synthesized.

Compositions According to the Invention

In another aspect, the invention includes a composition, which is secreted by the pituitary gland. Such a composition is associated with obesity and its expression status is modulated by leptin treatment. By “expression status” is meant the degree to which the composition is expressed. In specific embodiments, this composition is selected from the group consisting of OBs:1-6. This composition can be used in any of the methods according to the invention.

The invention will be further described in the following examples, which do not limit the scope of the invention described in the claims.

### EXAMPLE 1

### Animals

The four groups of mice, which were treated to generate the samples for GeneCalling were:
A) obese (ob/ob) treated with phosphate buffered saline (PBS) (“vehicle”) [“obese/PBS”]
B) obese (ob/ob) treated with leptin-IgG/PBS (“leptin”) [“obese/leptin”]
C) lean (ob/+ or +/+) treated with PBS [“lean/PBS”]
D) lean (ob/+ or +/+) treated with leptin-IgG/PBS [“lean/leptin”]

Mice (C57B1/6 obese (ob/ob) and lean (ob/+, +/+littermates; 8 weeks of age; females) were purchased from the Jackson Labs. They were acclimated for ten days prior to the beginning of the experiment. Food and water were provided ad. lib. and they were maintained on a 12 hour (6:00 pm to 6:00 am) dark:light cycle. Each mouse was weighed and dosed at 1 mg/kg with a leptin-IgG fusion protein. The mice were injected daily and weights and food intake were measured. Weight gain over the seven days was: obese/PBS, 2.7+/−0.3 gms; obese/leptin, −3.5+/0.2 gms; lean/PBS 0.6+/−0.1 gms; lean/leptin, −0.4+/−0.1 gm. Food intake (per mouse over 7 days) was: obese/PBS, 35+/−0.8 gms; obese/leptin, 20+/−0.5 gms; lean/PBS 23+/−0.8 gms; lean/leptin, 18.5+/−0.5 gm.

After seven daily injections the mice were sacrificed and muscle (gastrocnemius), liver, fat (pooled peri-renal and ovarian), pituitaries and hypothalami were removed and snap frozen on dry ice. RNA was extracted from the tissues as described below. A total of 240 mice (120 obese and 120 lean) in four groups were used. For each tissue three pools were prepared with each pool containing tissue from between five (liver) and twenty (pituitary and hypothalamus) mice. RNA was prepared from each pool of tissue for GeneCalling®.

### EXAMPLE 2

### Differential Gene Expression Analysis

GeneCalling® reactions were performed essentially as described (Shimkets et al., Nature Biotechnology 17:798-803 (1999)). In brief, total cellular RNA was isolated with Trizol (BRL, Grand Island N.Y.) using one-tenth volume of bromochloropropane for phase separation (Molecular Research Center Inc., Cincinnati Ohio). Contaminating DNA was removed by treatment with DNAse I (Promega, Madison Wis.) in the presence of 0.01 M DTT (BRL, Grand Island N.Y.) and 1 unit/μl RNasin (Promega, Madison Wis.). Following phenol/chloroform extraction, RNA quality was evaluated by spectrophotometry and formaldehyde agarose gel electrophoresis, and RNA yield was estimated by fluorometry with OliGreen (Molecular Probes, Eugene Oreg.). Poly-A+ RNA was prepared from 50 μg total RNA using oligo(dT) magnetic beads (PerSeptive, Cambridge Mass.), and quantitated with fluorometry.

First strand cDNA was prepared from 1.0 μg of poly(A)+using Superscript II reverse transcriptase (BRL, Grand Island N.Y.). Second strand synthesis was performed following the addition of E. coli DNA ligase, E. coli DNA polymerase, and E. coli RNase H (all from BRL, Grand Island N.Y.). Five units of T4 DNA polymerase was then added and the incubation continued for 5 minutes at 16° C. The reaction was treated with arctic shrimp alkaline phosphatase (USB, Cleveland Ohio), and cDNA purified by phenol/chloroform extraction. The yield of cDNA was estimated using fluorometry with PicoGreen (Molecular Probes, Eugene Oreg.).

### EXAMPLE 3

### cDNA Fragmentation, Tagging and Amplification

Fragmentation was achieved by 96 individual restriction enzyme digestions, each using 1 of 96 restriction enzyme pairs. Tagging was achieved by T4 DNA ligase reactions of amplification cassettes with ends compatible to the 5′ and 3′ ends of the cDNA fragments generated during restriction digests. Amplification cassettes cause one strand of the PCR product to be labeled with a fluorescent tag (FAM) and the other with a biotin. Amplification was performed using a combination of Klentaq (Clontech Advantage) and PFU (Stratagene, La Jolla Calif.) thermostable polymerases. PCR product purification was performed using magnetic streptavidin beads (CPG Inc., Lincoln Park N.J.) and a magnet. Purified fluorescently labeled and biotinylated PCR products bound to magnetic beads were denatured in gel loading buffer containing a ROX-tagged molecular size standard (PE-Applied Biosystems, Foster City Calif.) under conditions that solublize the FAM-labeled cDNA strand while allowing the biotinylated strand to remain associated with the streptavidin-coated MPG™ magnetic beads. Following denaturation samples were loaded onto 5% polyacrylamide, 6M urea, 0.5×TBE ultrathin gels and electrophoresed on a Niagara instrument. PCR products are visualized by virtue of the fluorescent label at the 5′ end of one of the PCR primers that is not biotinylated, thus ensuring that all detected fragments have been digested by both enzymes.

The primary components of the Niagara gel electrophoresis system are a horizontal ultrathin 48 well gel cassette mounted in a platform employing stationary laser excitation and a multi-color CCD imaging system making possible the detection of the purified fluorescently-labeled, single-stranded, cDNA fragments generated as described above.

### EXAMPLE 4

### Gel Interpretation

The output of the electrophoresis instruments is processed using the Java-based, internet-ready, Open Genome Initiative (OGI) software suite. Each lane contains the fluorescently-labeled products of a single GeneCalling reaction plus the sizing ladder spanning the range from 50 to 500 bp. The ROX ladder peaks provide a correlation between CCD camera frames and DNA fragment size in base pairs. The OGI software is used to track the lanes and the signal generated by the ROX and FAM peaks from the sizing ladder and samples, respectively. Linear interpolation between the ladder peaks is used to convert the fluorescence traces from frames to base pairs. Data, corresponding to FAM labeled ssDNA that is now both sized and with 5′ and 3′ ends defined by the restriction pairs used to digest the cDNA, are submitted as point-by-point length versus amplitude addresses to an Oracle 8 database.

### EXAMPLE 5

### Differential Expression Analysis and Provisional Gene Identification

For each cDNA pool generated from each of three tissue samples, three independent GeneCalling reactions were performed. Composite traces representing each sample for the OGI-generated trace data from the GeneCalling reactions were compared pair-wise (i.e., ob/ob pituitary±leptin) using software designed to detect difference over certain threshold limits. Database queries were performed using the information inherent to the sized fragments with ends defined by restriction digest fragmentation.

### EXAMPLE 6

### Gene Confirmation by Oligonucleotide Poisoning

Restriction fragments that map in end sequence and length to known mouse genes were used as templates for the design of unlabeled oligonucleotide primers. An unlabeled oligonucleotide designed against one end of the restriction fragment was added in excess to the original reaction, and is re-amplified by PCR. This new reaction with the competing PCR primer was then electrophoresed and compared to a control reaction reamplified without the unlabeled oligonucleotide to evaluate the selective diminution of the peak of interest. See Shimkets et al., supra.

### EXAMPLE 7

### Real Time Quantitative PCR (“RTQ-PCR”)

RTQ-PCR was performed using an ABI PRISM 7700 Sequence Detection System instrument and software (PE Applied Biosystems, Inc., Foster City, Calif.) as described using the primers described in Table 3. See Heid et al., Genome Res. 6:986-94 (1996) and Gibson et al., Genome Res. 6:995-1001 (1996).

TABLE 3

| | | |
|---|---|---|
| Gene | Direction | Sequence |

| | | | |
|---|---|---|---|
| RPL19 | Forward | 5p ATGTATCACAGCCTGTACCTG | (SEQ ID NO:2) |
| | Reverse | 5p TTCTTGGTCTCTTCCTCCTTG | (SEQ ID NO:3) |
| | probe | 5p AGGTCTAAGACCAAGGAAGCACGCAA | (SEQ ID NO:4) |
| POMC | forward | 5p AGCAACCCGCCCAAGG | (SEQ ID NO:5) |
| | reverse | 5p GCGTCTGGCTCTTCTCGG | (SEQ ID NO:6) |
| | probe | 5p CAAGCGTTACGGTGGCTTCATGACC | (SEQ ID NO:7) |
| PC2 | forward | 5p CAGACCAGCGAATAACAAGCG | (SEQ ID NO:8) |
| | reverse | 5p GAAGCCGAGGTGCCTGTGT | (SEQ ID NO:9) |
| | probe | 5p TGACCTGCACAATGACTGCACAGAGACC | (SEQ ID NO:10) |
| Prolactin | forward | 5p CCTGCTGTTCTGCCAAAATGT | (SEQ ID NO:11) |
| | reverse | 5p TCGGAGAGAAGTCTGGCAGTC | (SEQ ID NO:12) |
| | probe | 5p AGCCTCTGCCAATCTGTTCCGCTG | (SEQ ID NO:13) |
| Leptin Rec. | forward | 5p TTGTTTTGTGGACCACCGAA | (SEQ ID NO:14) |
| | reverse | 5p TCAAAGCCGAGGCATTGTTT | (SEQ ID NO:15) |
| | probe | 5p CAACCGATGACTCCTTTCTCTCACCTGCT | (SEQ ID NO:16) |
| HSG P25L2G_1 | forward | 5p AACTCTGGTTCCCTTGAAGAAAATATT | (SEQ ID NO:17) |
| | Reverse | 5p GTGAGTATGCCTACCAAATGTTGTG | (SEQ ID NO:18) |
| | probe | 5p AGGTGTGGTGACGCCTGCCTCTTTAA | (SEQ ID NO:19) |

### EXAMPLE 8

### Adipocyte Cultures

Adipocytes were prepared from ovarian fat pads of 8 week old fasted (2 hours) female C57B1/6J mice (Jackson Labs, Bar Harbor, Me.). See Rodbell et al., J. Biol. Chem. 239:375-80 (1964). Fat pads were minced in Krebs-Ringer HEPES buffer (pH 7.4 containing 200 nM adenosine, 5 mM glucose, 3% fraction V BSA, 135 mM NaCl, 2.2 mM CaCl₂, 1.25 mM MgSO4, 0.45 mM KH2PO4, 2.17 mM Na2HPO₄, and 10 mM HEPES). Adipose tissue fragments were digested in the same buffer in the presence of type I collagenase (1 mg/ml; Worthington Biochemical, Lakewood, N.J.) at 37° C. with gentle shaking (100 rpm) for 30 minutes. Isolated adipocytes were separated from undigested tissue by filtration through a 250 uM polypropylene mesh and washed three times. For washing, cells were centrifuged at 500 rpm for. 3 minutes. Each time the infranatant was discarded and cells were resuspended in Krebs-Ringer HEPES buffer with the final wash being in 5.5 mM glucose DMEM, with 5% BSA, 20 mM HEPES, 1U/ml adenosine deaminase and 10 pM (−)—N-6-(2-phenylisopropyladenosine). Adipocytes were cultured in 48-well plates, 5×105 cells per 500 ul per well. Cells were treated in quadruplicate with rat ACTH (100, 10, 1 and 0.1 nM; Sigma) or rat prolactin (100, 10, 1 and 0.1 nM; Accurate Chemical). rhInsulin (1, 0.1 and 0.01 nM; Genentech) and isoproterenol (30, 10, 3 and 1 nM; Sigma) were added to separate wells as positive controls. Cells were incubated at 37° C./5% CO2. 50 ul was sampled at 4 h and again at 16 h. Media glucose was measured by a hexokinase colorimetric assay (Sigma), media glycerol was measured by a glycerol kinase/oxidase colorimetric assay (Sigma) and media leptin was measured by ELISA (Crystal Chem). RNA was prepared from the adipocytes using commercially available material and protocols (RNA STAT).

### EXAMPLE 9

### Pituitary Cultures

Pituitary cell cultures were prepared from whole pituitaries of 8 week old female C57B1/6J mice. Pituitaries were finely minced and then digested by rapid agitation for 30 minutes at 37° C. in Hank's balanced salt solution containing 4 mg/ml collagenase (type 2, CLS 2, Worthington) and 400 ug/ml DNase. Digested pituitaries were washed twice in low glucose DMEM with 10% FBS and then plated 300,000 cells per well in laminin coated 6-well plates. Cells were incubated at 37° C. C/5% CO₂ overnight before being treated with leptin. Murine leptin (BIOMOL) was added in quadruplicate (100, 10 and 1 nM). RNA was prepared from the pituitary using commercially available material and protocols (RNA STAT).

### EXAMPLE 10

### Quantitative Expression Analysis

The effect of obesity and leptin administration on gene expression differences was examined using Quantitative Expression Analysis (QEA™). Five of the major tissues implicated in metabolic control (pituitary, hypothalamus, muscle, liver and fat) were analyzed from each of four groups of female mice: obese (ob/ob) treated with leptin; obese treated with vehicle (PBS); lean (ob/+ or +/+) treated with leptin and lean treated with vehicle. For each tissue three pools were prepared with each pool containing tissue from between five (liver) and twenty (pituitary and hypothalamus) mice. RNA was prepared from each pool of tissue and the cDNA derived from the RNA was analyzed using 96 pairs of restriction enzymes. Previous results indicate that this will allow the analysis of greater than 90% of the expressed genome with a sensitivity of detection greater than 1:100,000 (Shimkets et al., supra). Three binary comparisons were initially made—obese versus lean mice (both vehicle treated), lean mice, vehicle versus leptin treated and obese mice leptin versus vehicle treated. A difference was called if the peak heights differed by more than two fold (p<0.05). The results of these comparisons are shown in Table 4. Each gene fragment represents part of a gene and any one gene has the potential for generating multiple independent gene fragments—some genes will give rise to only one that is detectable while others can give rise to five to ten. There is an approximate three to one ratio between gene fragments and represented genes. That the differences in peak height reflect a difference in expression of the underlying mRNA has been previously validated (Shimkets et al., supra).

RNA from each of the five tissues was analyzed by QEA and the number of detectable gene fragments determined (Table 4). As described above, each gene fragment represents part of a particular cDNA and so the number of gene fragments found can be used as a measure of the number of genes expressed within the tissue. There is a large difference in the number of gene fragments detected in the fat as compared to the other tissues suggesting that fat expresses fewer genes compared to the other tissues. By comparing peak heights for each gene fragment, it is also apparent that there are large differences in the number of genes that are responding to either of obesity or leptin in the different tissues. Thus, the liver is very sensitive to obesity with 587 gene fragments (2.3% of the total) changing more than two fold relative to the expression in lean liver. In contrast only 82 (0.3%) differences were detected in the hypothalamus. The other tissues are intermediate between these two, with 117 (0.5%) gene fragments changing in the pituitary, 158 (0.6%) in muscle and 196 (1.6%) in fat. It should be noted that these assessments are drawn from the expression of the total tissue. As the liver is more homogenous than the hypothalamus in terms of cell type, the numbers of gene expression differences in the hypothalamus could be an underestimate. Fat and liver were the most responsive tissues to leptin treatment with approximately 0.6% and 0.4% of the genes changing more than two fold in response to a one week treatment. As described below this analysis does not address whether these are direct effects of leptin on the fat or liver as compared to leptin altering liver gene expression indirectly via, for example, alterations in proteins being delivered by the pituitary. There are more differences detected in response to leptin in the obese mice as compared to lean mice. This is comparable to what is seen with respect to the physiological responses (food intake and fat loss) in the same sets of mice.

The primary goal was the identification of those genes that are relevant to the development of obesity. The simple two way comparisons described above will identify not only these more relevant genes but also those that are altered as a compensatory response to obesity and those that are altered by leptin but may be related to the reproductive effects of leptin (Clarke et al., Reviews of Reproduction 4(1):48-55 (1999)). Thus the gene expression changes were further analyzed by searching for those genes for which expression was altered by obesity and at the same time expression was returned toward the lean pattern of expression by a one week course of treatment with leptin. Only the obese mice treated with leptin were used for this comparison as they are more sensitive to the effect of leptin.

Between 6% and 12% of the gene fragments that differ between obese and lean mice are at least partially normalized by leptin treatment (Table 4). Because of uncertainties relating to cellular heterogeneity and the relationship between gene fragment number and gene number it is unclear if the differences between the tissues are significant. The converse of this analysis indicates that approximately 90% of the obesity related differences are not significantly normalized by a one week course of treatment with leptin and points to the dangers inherent in a simple binary comparison for the detection of leptin responsive genes. Possible reasons for the failure of 90% of the differences to normalize would include the length of treatment and irreversible alterations established by eight weeks of leptin deficiency.

TABLE 4

| | Number of gene fragments | | | Gene fragments |
|---|---|---|---|---|
| | that are different in | | | altered by |
| | the indicated comparisons. | | | obesity and at |
| Tissue | Obese | Leptin vs. | Leptin vs. | least partially |
| (total number of gene | vs. | vehicle | vehicle | normalized |
| fragments analyzed) | Lean | (lean) | (obese) | by leptin. |
| Pituitary | 117 | 10 | 23 | 14 |
| (22,000) | | | | |
| Fat | 196 | 22 | 80 | 12 |
| (12,250) | | | | |
| Muscle | 158 | 29 | 44 | 18 |
| (26,300) | | | | |
| Liver | 587 | 32 | 110 | 58 |
| (25,520) | | | | |
| Hypothalamus | 82 | 32 | 73 | 5 |
| (27,000) | | | | |

The gene expression differences detected in the pituitary were further analyzed. Gene profiling allowed the identification of 117 gene fragments that were differentially expressed in the pituitaries of lean in comparison to obese mice. The minimum expression difference is 2 fold; the maximal differences can not be accurately estimated owing to the low level of expression of some genes. Based on the ratio of gene fragments identified to genes represented (Table 5), it is estimated that the 117 gene fragments represent approximately 40 different genes. A comparable assessment indicates that we could detect the expression of approximately 7 pituitary expressed genes that are regulated by leptin (in obese mice). Fourteen of the 117 gene fragments altered by obesity were at least partially normalized by leptin. These fourteen gene fragments are listed in Table 5 according to a nomenclature that is derived from the restriction fragments that were used to fragment the DNA and the size (in base pairs) of the fragment.

TABLE 5

| | Gene fragment | | |
|---|---|---|---|
| | identification | fold change in | fold change induced |
| Gene | number ID | obese vs. lean | by leptin |
| PC2 (OB1) | d010-154 | 7.8 | 0.62 |
| | l0e1-277 | 2.1 | 0.71 |
| | i0p0-185 | 4.2 | 0.59 |
| | i0u0-303 | 3.7 | 0.55 |
| Unknown | g1n0-246 | 0.09 | 2.68 |
| Novel (OB6) | i0m0-307 | 2.6 | 0.75 |
| Unknown | y0k0-253 | 2.8 | 0.59 |
| Unknown | i0n0-172 | 0.11 | 2.15 |
| Prolactin (OB3) | m1l0-118 | 0.31 | 1.73 |
| | r0v0-237 | 0.17 | 2.08 |
| | m1s0-329 | 0.35 | 1.41 |
| | r0y0-158 | 0.16 | 3.42 |
| POMC (OB2) | m0r0-191 | 5.9 | 0.5 |
| HSGP25L2G_1 | d0l0-136 | 6.5 | 0.58 |
| (OB5) | | | |

Gene identification of the 14 pituitary-derived gene fragments altered by both obesity and leptin treatment was carried out using a combination of oligonucleotide poisoning and gene fragment cloning and sequencing. By these approaches gene identification was determined for 10 of the 14 gene fragments. Four of the gene fragments correspond to the mRNA encoding the processing enzyme PC2 (OB1); four gene fragments correspond to the mRNA encoding prolactin (OB3), one gene fragment corresponds to the mRNA encoding the prepro-opiomelanocortin (POMC) (OB2) and one gene fragment corresponds to the mRNA encoding the mouse homologue of the protein HSGP25L2G_{—}1 (OB5). This protein is one of a closely related family of proteins that appear to reside within the endoplasmic reticulum but has an unknown function. Interestingly, the mRNA encoding one member of this family is co-ordinately expressed with POMC in Xenopus (Seidah et al., DNA & Cell Biology 9(6):415-24 (1990)). Identification of three gene fragments has remained undetermined and for one gene fragment, (i0m0-307) (OB6), the DNA sequence of the gene fragment was obtained but this does not contain any significant open reading frames and we have yet to identify a full length cDNA.

POMC/PC2 (OB2/OB1)

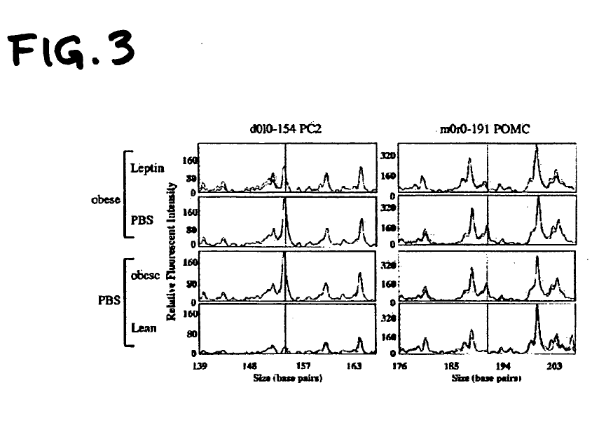

The QEA traces for one gene fragment corresponding to each of PC2 and POMC are shown in FIG. 3. Each of the four panels represents a comparison between either obese mice treated with leptin or PBS (upper panels) or between obese and lean mice treated with PBS (lower panels). Each data set is shown in a separate window and contains two or three traces. In some sets, only two traces are shown as not all cDNA was successfully analyzed for each pair of restriction fragments—a minimum of two traces was required for subsequent analysis. Each trace is derived from one of the three independent pools of pituitaries. Each cDNA sample was analyzed in triplicate and each trace represents the average of this triplicate. In FIG. 3, the relevant portion of the traces are shown (with the gene fragment size in base pairs shown under the trace) and the gene fragment that is different between the two samples is indicated by a vertical line. Note that within each window, the traces derived from independent pools of pituitaries largely overlap demonstrating the reproducibility of the technology. It is also clear that with the exception of the indicated target gene fragment the traces obtained from the different groups of mice are similar. For quantitation the gene fragment heights are calculated and the means and standard deviations for each gene fragment are calculated. For two genes (PC2 and prolactin) there are multiple gene fragments derived from each of the corresponding cDNAs that were altered in the same direction and to a comparable extent. This increases the confidence in both the identification of the gene underlying the gene fragment and the magnitude of the gene expression difference.

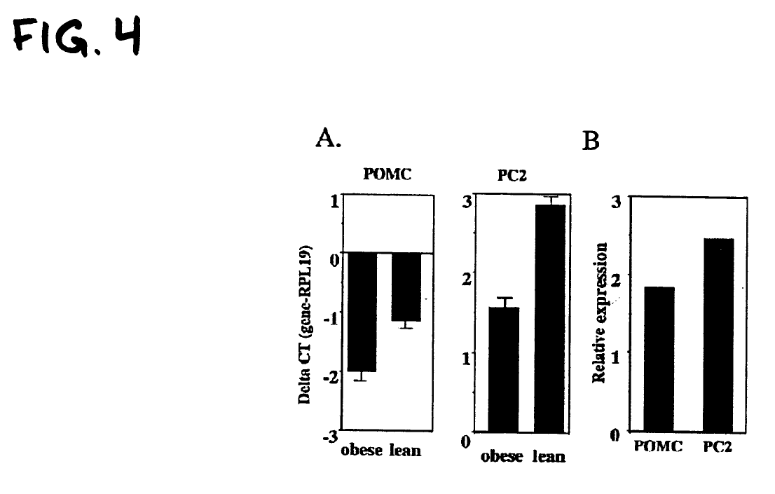

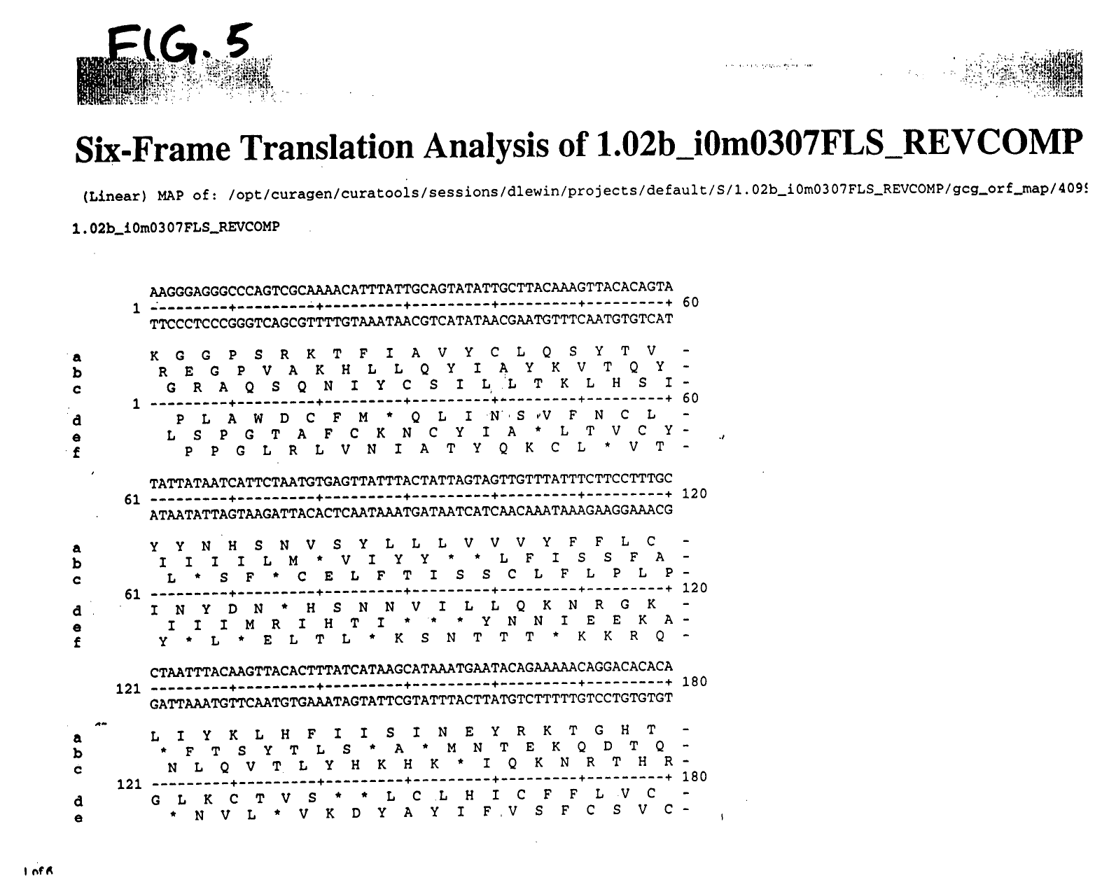

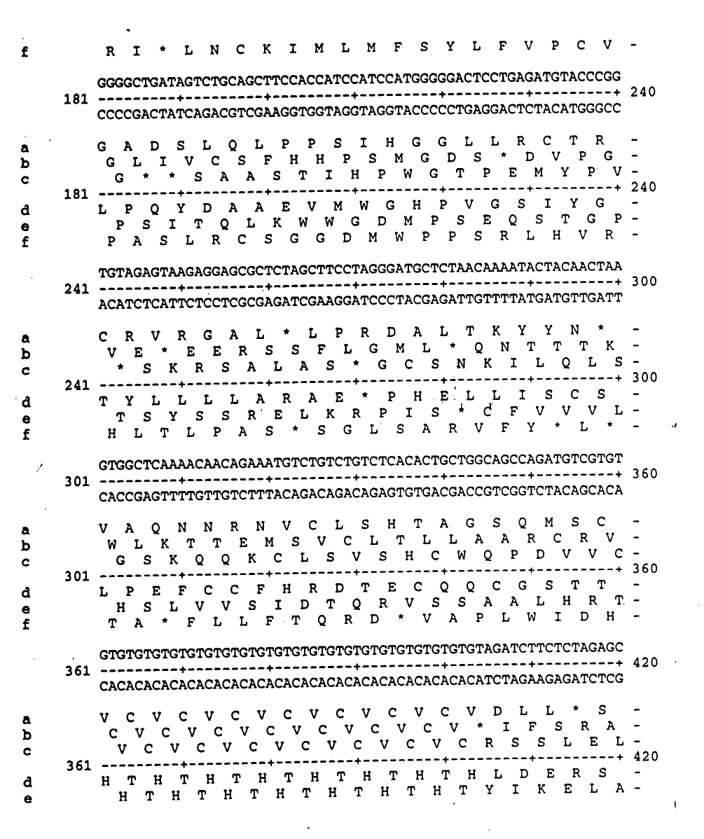

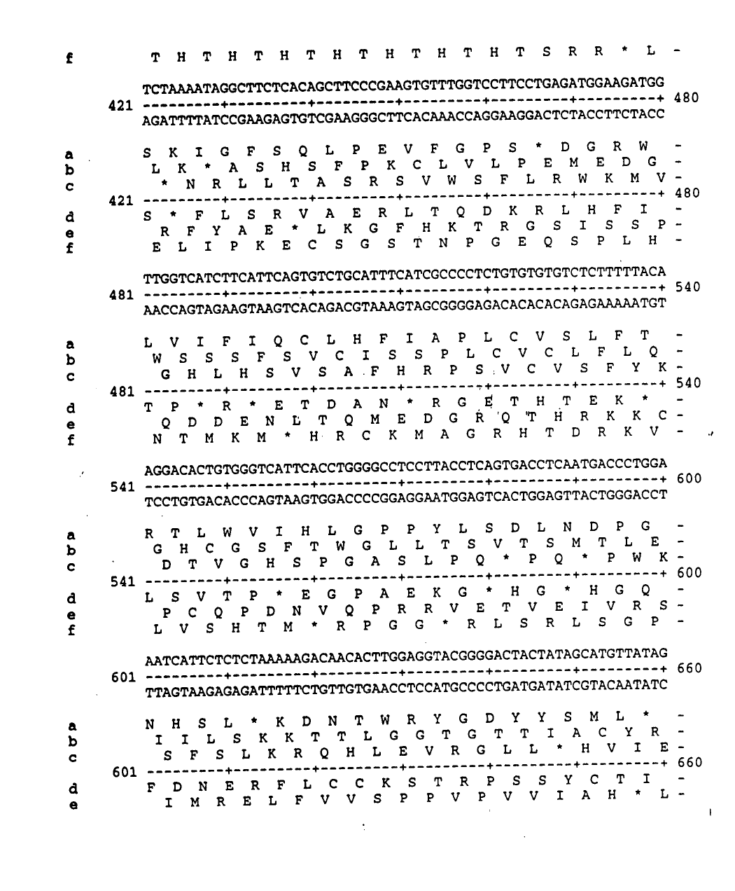

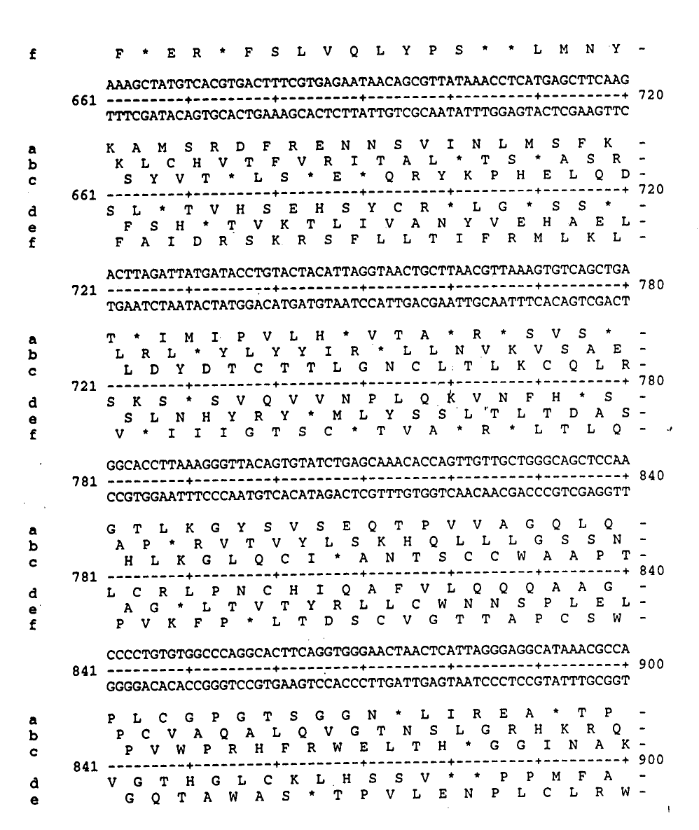

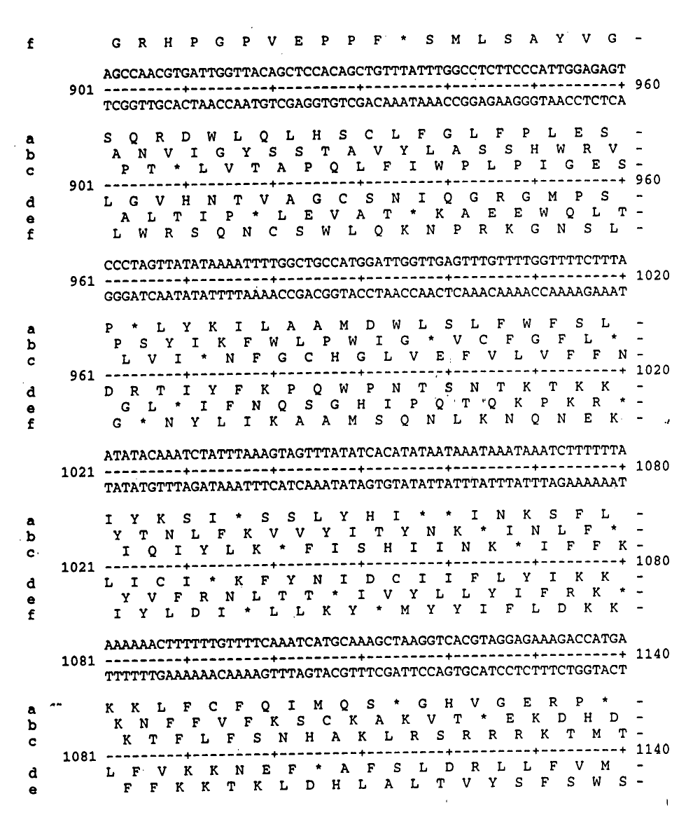

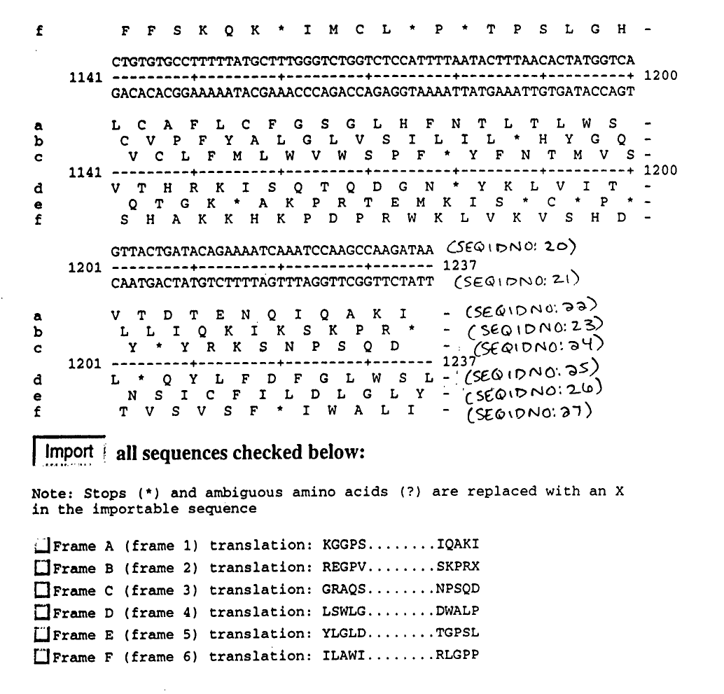

The reliability of QEA to detect gene expression differences has been previously documented (Shimkets et al., supra). To further increase confidence in the data set we have used real time quantitative PCR to characterize transcript levels for a representative set of the genes (PC2 and POMC). Again, three pools of pituitaries were used (completely independent of the original experiment and containing five pituitaries/pool); the extracted RNA was analyzed using real time quantitative PCR. The results shown in FIG. 4. confirm that obesity decreases the expression of both PC2 and POMC. The delta CT values are given in A and these data are used to generate the average relative expression (B).

The mRNA levels of both PC2 and POMC are increased in obese mice and decreased by leptin treatment. PC2 (See Bertagna, Endocrinol and Metabolism Clinics of North America 23(3):467-85 (1994)) is one of the two major proteases that appear to be involved in processing the POMC precursor to smaller bioactive peptide hormones. It is not yet know whether the effect of leptin on PC2 expression is limited to particular cell types. With the exception of ACTH, the physiological function of the other POMC derived peptides is still unclear. The possibility that a leptin mediated differential processing of the POMC precurser could be physiologically relevant remains to be addressed.

Prolactin (OB3)

Four gene fragments derived from the prolactin cDNA were altered by both obesity and by leptin treatment. Thus, obesity suppressed prolactin mRNA levels by three to five fold and leptin increased leptin mRNA levels by two to three fold in the obese mice (Table 5). That prolactin expression is both reduced by obesity and induced by leptin as reported here is consistent with a causal role for diminished prolactin in the development or maintenance of obesity. The mechanism(s) by which lower prolactin levels could contribute to the development of obesity are not clear.

HSGP25L2G^{—}1 (OB5)

The fourth gene identified is the mouse homologue of HSGP25L2G_{—}1. The expression of this gene was increased approximately six fold in obese mice and suppressed by 50% with leptin treatment. The encoded protein is one member of a family of proteins that appear to reside within the endoplasmic reticulum but have an unknown function. (See Wada et al., J. Biol. Chem. 266(29):19599-610 (1991)). Interestingly the mRNA encoding one member of this family is co-ordinately expressed with POMC in Xenopus. (See Holthuis et al., Biochem. J. 312 (Pt 1):205-13 (1995).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. ADA Report, Position of the American Dietetic Association: weight management. J Am Diet Assoc, 1997. 97(1): p. 71-4.
2. Pi-Sunjer, F. and N.O.E.I.E. Panel, Clinical Guidelines on the Identification, Evaluation, and Treatment of Overweight and Obesity in Adults—The Evidence Report. NIH Publication No. 98-4083 ed. 1998: National Institutes of Health.
3. Perusse, L. and C. Bouchard, Role of genetic factors in childhood obesity and in susceptibility to dietary variations. Ann Med, 1999. 31 Suppl 1: p. 19-25.
4. Dunlop, J. and S. Rosenzweig-Lipson, Therapeutic approaches to obesity. Exp. Opin. Ther. Patents, 1998. 8(12): p. 1683-1694.
5. Bouchard, C., Genetics of obesity: an update on molecular markers. Int J Obes Relat Metab Disord, 1995. 19 Suppl 3: p. S10-3.
6. Weigle, D. S. and J. L. Kuijper, Obesity genes and the regulation of body fat content. Bioessays, 1996. 18(11): p. 867-74.
7. Spiegelman, B. M. and J. S. Flier, Adipogenesis and obesity: rounding out the big picture. Cell, 1996. 87(3): p. 377-89.
8. Montague, C. T., et al., Congenital leptin deficiency is associated with severe early-onset obesity in humans. Nature, 1997. 387(6636): p. 903-8.
9. Clement, K., et al., A mutation in the human leptin receptor gene causes obesity and pituitary dysfunction [see comments]. Nature, 1998. 392(6674): p. 398-401.
10. Wexler, B. C., S. G. Iams, and J. P. McMurtry, Pathophysiological differences between obese and non-obese spontaneously hypertensive rats. Br J Exp Pathol, 1980. 61(2): p. 195-207.
11. Van Zwieten, P. A., et al., Hypertensive diabetic rats in pharmacological studies. Pharmacol Res, 1996. 33(2): p. 95-105.
12. Pomp, D., Genetic dissection of obesity in polygenic animal models. Behav Genet, 1997. 27(4): p. 285-306.
13. Aron, D., J. Findling, and J. Tyrell, Hypothalamus & Pituitary, in Basic & Clinical Endocrinology, F. Greenspan and G. Strewler, Editors. 1997, Appleton & Lange: Stamford, Conn. p. 95-156.
14. Guan, X. M., H. Yu, and L. H. Van der Ploeg, Evidence of altered hypothalamic pro-opiomelanocortin/neuropeptide Y mRNA expression in tubby mice. Brain Res Mol Brain Res, 1998. 59(2): p. 273-9.
15. Yaswen, L., et al., Obesity in the mouse model of pro-opiomelanocortin deficiency responds to peripheral melanocortin. Nat Med, 1999. 5(9): p. 1066-1070.
16. Tartaglia, L. A., Polypeptides involved in body weight disorders, including obesity, in U.S. Patent. 1999, Millenium Pharmaceuticals, Inc.: United States.
17. Wang, J., et al., A nutrient-sensing pathway regulates leptin gene expression in muscle and fat. Nature, 1998. 393(6686): p. 684-8.
18. Mulvihill, S. and H. Debas, Regulatory Peptides of the Gut, in Basic & Clinical Endocrinology, F. Greenspan and G. Strewler, Editors. 1997, Appleton & Lange: Stamford, Conn. p. 575-594.
19. Bado, A., et al., The stomach is a source of leptin. Nature, 1998. 394(6695): p. 790-3.
20. Lord, G. M., et al., Leptin modulates the T-cell immune response and reverses starvation-induced immunosuppression. Nature, 1998. 394(6696): p. 897-901.
21. Wade, G. N., et al., Leptin facilitates and inhibits sexual behavior in female hamsters. Am J Physiol, 1997. 272(4 Pt 2): p. R1354-8.
22. Considine, R. V. and J. F. Caro, Leptin and the regulation of body weight. Int J Biochem Cell Biol, 1997. 29(11): p. 1255-72.
23. Friedman, J. M. and J. L. Halaas, Leptin and the regulation of body weight in mammals. Nature, 1998. 395(6704): p. 763-70.
24. Aron, D. C., J. W. Findling, and J. B. Tyrell, Hypothalamus and Pituitary, in Basic & Clinical Endocrinology, F. S. Greenspan and G. J. Strewler, Editors. 1997, Appleton & Lange: Stamford. p. 95-156.
25. Leighton, P. A., et al., Genomic imprinting in mice: its function and mechanism. Biol Reprod, 1996. 54(2): p. 273-8.
26. Barsh, G., From Agouti to Pomc—100 years of fat blonde mice [news; comment]. Nat Med, 1999. 5(9): p. 984-5.
27. Dietrich, J. B., Neuropeptides, antagonists and cell proliferation: bombesin as an example. Cell Mol Biol (Noisy-le-grand), 1994. 40(6): p. 731-46.
28. Henkel, M., et al., Endocytic delivery of intramolecularly quenched substrates and inhibitors to the intracellular yeast Kex2 protease1. Biochem J, 1999. 341(2): p. 445-52.

1. A method of assessing the efficacy of an obesity treatment in a subject, the method comprising:
a) providing from the subject a test cell population comprising cells capable of expressing one or more nucleic acid sequences selected from the group consisting of OB1-6;
b) detecting expression of one or more of the nucleic acid sequences in said test cell population;
c) comparing the expression of the nucleic acid sequences in the test cell population to the expression of the nucleic acid sequences in a reference cell population comprising at least one cell whose obesity stage is known; and
d) identifying a difference in expression levels of the OB1-6 sequences, if present, in the test cell population and the reference cell population,
thereby assessing the efficacy of an obesity treatment in the subject.

2. The method of claim 1, wherein the subject is a mammal.

3. The method of claim 2, wherein the subject is human.

4. The method of claim 1, wherein the method comprises comparing the expression of two or more of the nucleic acid sequences.

5. The method of claim 1, wherein the method comprises comparing the expression of four or more of the nucleic acid sequences.

6. The method of claim 1, wherein the method comprises comparing the expression of six or more of the nucleic acid sequences.

7. The method of claim 1, wherein the expression of the nucleic acid sequences in the test cell population is increased as compared to the reference cell population.

8. The method of claim 1, wherein the test cell population is provided in vitro.

8. The method of claim 1, wherein the test cell population is provided ex vivo from a mammalian subject.

9. The method of claim 1, wherein the test cell is provided in vivo in a mammalian subject.

10. A method of identifying a test therapeutic agent for treating obesity in a subject, the method comprising:
a) providing from the subject a test cell population comprising cells capable of expressing one or more nucleic acid sequences selected from the group consisting of OB1-6;
b) contacting said test cell population with the test therapeutic agent;
c) detecting the expression of one or more of the nucleic acid sequences in said test cell population;
d) comparing the expression of the nucleic acid sequences in the test cell population to the expression of the nucleic acid sequences in a reference cell population comprising at least one cell whose obesity stage is known; and
e) identifying a difference in expression levels of the OB1-6 sequences, if present, in the test cell population and the reference cell population, thereby identifying a test therapeutic agent for treating obesity in a subject.

11. The method of claim 10 wherein the subject is a mammal.

12. The method of claim 11 wherein the subject is human.

13. The method of claim 10 wherein the test therapeutic agent is a known anti-obesity agent.

14. The method of claim 13 wherein the test therapeutic agent is selected from the group consisting of: dexfenfluramine, sibutramine, beta3-adrenergic agonists, and olistat.

15. The method of claim 10 wherein the test therapeutic agent is an unknown anti-obesity agent.

16. A method of identifying leptin-induced nucleic acid sequences, the method comprising:
a) providing a test cell population from a subject whose obesity stage is known;
b) measuring the expression of one or more nucleic acid sequences expressed by the test cell population;
c) comparing the expression of the nucleic acid sequences in the test cell population to the expression of the nucleic acid sequences in a reference cell population comprising at least one cell from a subject with the opposite obesity stage;
d) determining which nucleic acid sequences, if any, are differentially expressed in the test cell population and the reference cell population;
e) adding leptin to the test cell population and the reference cell population;
f) comparing the expression of the nucleic acid sequences in the test cell population after leptin treatment with the expression of the nucleic acid sequences in the reference cell population after leptin treatment; and
g) identifying a difference in expression levels, if present, in the test cell population and the reference cell population,
thereby identifying leptin-induced nucleic acid sequences.

17. A method of diagnosing or determining the susceptibility to obesity in a subject, the method comprising:
a) providing from the subject a test cell population comprising cells capable of expressing one or more nucleic acid sequences selected from the group consisting of OBs:1-6;
b) measuring expression of one or more of the nucleic acid sequences in the test cell population;
c) comparing the expression of the nucleic acid sequences in the test cell population to the expression of the nucleic acid sequences in a reference cell population comprising at least one cell from a subject not suffering from obesity; and
d) identifying a difference in expression levels of the nucleic acid sequences, if present, in the test cell population and reference cell population,
thereby diagnosing or determining the susceptibility to obesity in the subject.

18. The method of claim 17 wherein the subject is a mammal.

19. The method of claim 18 wherein the subject is a human.

20. A method of treating obesity, the method comprising administering to a patient suffering from or at risk for developing obesity, an agent that modulates the expression or activity of one or more nucleic acid sequences selected from the group consisting of OBs:1-6.

21. The method of claim 20, the method comprising administering to a patient suffering from or at risk for developing obesity, an agent that decreases the expression or activity of one or more nucleic acid sequences selected from the group consisting of OBs:1, 2, 4, 5, and 6.

22. The method of claim 20, the method comprising administering to a patient suffering from or at risk for developing obesity, an agent that increases the expression or activity of one or more nucleic acid sequences selected from the group consisting of OB:3.

23. The method of claim 20, wherein the agent is an antibody to a polypeptide encoded by the OB nucleic acid sequence, a peptide, peptidomimetic, small molecule, or other drug.

24. A kit comprising one or more reagents for detecting two or more nucleic acid sequences selected from the group consisting of OBs:1-6.

25. An array of probe nucleic acids, wherein said probe nucleic acids detect two or more nucleic acid sequences selected from the group consisting of OBs:1-6.

26. An isolated nucleic acid molecule comprising a nucleic acid sequence that is least 75% identical to SEQ ID NO:1, or the complement of the nucleic acid sequence.

27. A nucleic acid vector comprising the nucleic acid sequence of claim 26.

28. A host cell comprising the isolated nucleic acid molecule of claim 26.

29. An isolated polypeptide at least 80% identical to a polypeptide selected from the group consisting of:
a) a polypeptide comprising an amino acid sequence encoded by the nucleic acid sequence of SEQ ID NO:1;
b) a fragment of a polypeptide comprising an amino acid sequence encoded by the nucleic acid sequence of SEQ ID NO:1, wherein the fragment comprises at least 6 contiguous amino acids;
c) a derivative of a polypeptide comprising an amino acid sequence encoded by the nucleic acid sequence of SEQ ID NO:1;
d) an analog of a polypeptide comprising an amino acid sequence encoded by the nucleic acid sequence of SEQ ID NO:1; and
e) a homolog of a polypeptide comprising an amino acid sequence encoded by the nucleic acid sequence of SEQ ID NO:1.

30. An antibody that selectively binds to the polypeptide of claim 29, and fragments, homologs, analogs and derivatives of the antibody.

31. A pharmaceutical composition comprising the nucleic acid of claim 26.

32. A pharmaceutical composition comprising the polypeptide of claim 29.

33. A method of detecting the presence of the nucleic acid of claim 26 in a sample, comprising contacting the sample with a compound that selectively binds to the nucleic acid of claim 26 and determining whether the compound bound to the nucleic acid of claim 26 is present in the sample.

34. A method for modulating the activity of the nucleic acid of claim 26, the method comprising contacting a cell sample comprising the nucleic acid of claim 26, with a compound that binds to said nucleic acid in an amount sufficient to modulate the activity of the polypeptide.

35. An isolated polypeptide used to treat obesity in a subject, wherein the polypeptide is at least 80% identical to a polypeptide selected from the group consisting of:
a) a polypeptide comprising an amino acid sequence of OBs:1-6;
b) a fragment of a polypeptide comprising an amino acid sequence of OBs:1-6, wherein the fragment comprises at least 6 contiguous amino acids of OBs:1-6;
c) a derivative of a polypeptide comprising an amino acid sequence of OBs:1-6;
d) an analog of a polypeptide comprising an amino acid sequence of OBs:1-6; and
e) a homolog of a polypeptide comprising an amino acid sequence of OBs:1-6.

36. The polypeptide of claim wherein the expression status of the polypeptide is regulated by leptin.

37. The polypeptide of claim 35, wherein the expression of the polypeptide is down-regulated by leptin and the polypeptide is selected from the group consisting of OBs:1, 2, and 4-6.

38. The polypeptide of claim 36, wherein the expression of the polypeptide is up-regulated by leptin and the polypeptide is selected from the groups consisting of OBs:3.

39. The polypeptide of claim 35, wherein the subject is a mammal.

40. The polypeptide of claim 39, wherein the subject is human.

41. The polypeptide claim 35, wherein the polypeptide is secreted from the pituitary gland.

42. An isolated nucleic acid molecule used to treat obesity in a subject, wherein the nucleic acid is least 75% identical to the nucleic acid sequence any one of OBs:1-6 or the complement of the nucleic acid sequence.

43. The nucleic acid molecule of claim 42, wherein the expression status of the nucleic acid sequence is regulated by leptin.

44. The nucleic acid molecule of claim 43, wherein the expression status of the nucleic acid sequence is down-regulated by leptin and the nucleic acid is selected from the group consisting of OBs:1,2, and 4-6.

45. The nucleic acid molecule of claim 43, wherein the expression status of the nucleic acid sequence is up-regulated by leptin and the nucleic acid is selected from the group consisting of OBs:3.

46. The nucleic acid molecule of claim 42 wherein the subject is a mammal.

47. The nucleic acid molecule of claim 42 wherein the subject is human.

48. The nucleic acid sequence of claim 42, wherein the nucleic acid sequence is expressed in the pituitary gland.

49. A composition which is secreted by the pituitary gland, is associated with obesity, and whose expression status is modulated by leptin.

50. The composition of claim 49, wherein the composition is selected from the group consisting of OBs:1-6.


SEQUENCE LISTING (27 sequences)

SEQ ID NO: 1 - DNA, 1238 bp, Mus sp.
ttatcttggcttggatttgattttctgtatcagtaactgaccatagtgttaaagtattaaaatggagacccagacccaaagcataaaaaggcacacagtcatggtctttctcctacgtgaccttagctttgcatgatttgaaaacaaaaaagtttttttaaaaaagatttatttatttattatatgtgatataaactactttaaatagatttgtatattaaagaaaaccaaaacaaactcaaccaatccatggcagccaaaattttatataactagggactctccaatgggaagaggccaaataaacagctgtggagctgtaaccaatcacgttggcttggcgtttatgcctccctaatgagttagttcccacctgaagtgcctgggccacacaggggttggagctgcccagcaacaactggtgtttgctcagatacactgtaaccctttaaggtgcctcagctgacactttaacgttaagcagttacctaatgtagtacaggtatcataatctaagtcttgaagctcatgaggtttataacgctgttattctcacgaaagtcacgtgacatagctttctataacatgctatagtagtccccgtacctccaagtgttgtctttttagagagaatgatttccagggtcattgaggtcactgaggtaaggaggccccaggtgaatgacccacagtgtccttgtaaaaagagacacacacagaggggcgatgaaatgcagacactgaatgaagatgaccaaccatcttccatctcaggaaggaccaaacacttcgggaagctgtgagaagcctattttagagctctagagaagatctacacacacacacacacacacacacacacacacacacacacacacacacgacatctggctgccagcagtgtgagacagacagacatttctgttgttttgagccacttagttgtagtattttgttagagcatccctaggaagctagagcgctcctcttactctacaccgggtacatctcaggagtcccccatggatggatggtggaagctgcagactatcagcccctgtgtgtcctgtttttctgtattcatttatgcttatgataaagtgtaacttgtaaattaggcaaaggaagaaataaacaactactaatagtaaataactcacattagaatgattataatatactgtgtaactttgtaagcaatatactgcaataaatgttttgcgactgggccctccctt

SEQ ID NO: 2 - DNA, 21 bp, Artificial Sequence
  Description of Artificial Sequence Primer
atgtatcacagcctgtacctg

SEQ ID NO: 3 - DNA, 21 bp, Artificial Sequence
  Description of Artificial Sequence Primer
ttcttggtctcttcctccttg

SEQ ID NO: 4 - DNA, 26 bp, Artificial Sequence
  Description of Artificial Sequence Probe
aggtctaagaccaaggaagcacgcaa

SEQ ID NO: 5 - DNA, 16 bp, Artificial Sequence
  Description of Artificial Sequence Primer
agcaacccgcccaagg

SEQ ID NO: 6 - DNA, 18 bp, Artificial Sequence
  Description of Artificial Sequence Primer
gcgtctggctcttctcgg

SEQ ID NO: 7 - DNA, 25 bp, Artificial Sequence
  Description of Artificial Sequence Probe
caagcgttacggtggcttcatgacc

SEQ ID NO: 8 - DNA, 21 bp, Artificial Sequence
  Description of Artificial Sequence Primer
cagaccagcgaataacaagcg

SEQ ID NO: 9 - DNA, 19 bp, Artificial Sequence
  Description of Artificial Sequence Primer
gaagccgaggtgcctgtgt

SEQ ID NO: 10 - DNA, 28 bp, Artificial Sequence
  Description of Artificial Sequence Probe
tgacctgcacaatgactgcacagagacc

SEQ ID NO: 11 - DNA, 21 bp, Artificial Sequence
  Description of Artificial Sequence Primer
cctgctgttctgccaaaatgt

SEQ ID NO: 12 - DNA, 21 bp, Artificial Sequence
  Description of Artificial Sequence Primer
tcggagagaagtctggcagtc

SEQ ID NO: 13 - DNA, 24 bp, Artificial Sequence
  Description of Artificial Sequence Probe
agcctctgccaatctgttccgctg

SEQ ID NO: 14 - DNA, 20 bp, Artificial Sequence
  Description of Artificial Sequence Primer
ttgttttgtggaccaccgaa

SEQ ID NO: 15 - DNA, 20 bp, Artificial Sequence
  Description of Artificial Sequence Primer
tcaaagccgaggcattgttt

SEQ ID NO: 16 - DNA, 29 bp, Artificial Sequence
  Description of Artificial Sequence Probe
caaccgatgactcctttctctcacctgct

SEQ ID NO: 17 - DNA, 27 bp, Artificial Sequence
  Description of Artificial Sequence Primer
aactctggttcccttgaagaaaatatt

SEQ ID NO: 18 - DNA, 25 bp, Artificial Sequence
  Description of Artificial Sequence Primer
gtgagtatgcctaccaaatgttgtg

SEQ ID NO: 19 - DNA, 26 bp, Artificial Sequence
  Description of Artificial Sequence Probe
aggtgtggtgacgcctgcctctttaa

SEQ ID NO: 20 - DNA, 1237 bp, Mus sp.
  CDS (1)..(1236)
aagggagggcccagtcgcaaaacatttattgcagtatattgcttacaaagttacacagtatattataatcattctaatgtgagttatttactattagtagttgtttatttcttcctttgcctaatttacaagttacactttatcataagcataaatgaatacagaaaaacaggacacacaggggctgatagtctgcagcttccaccatccatccatgggggactcctgagatgtacccggtgtagagtaagaggagcgctctagcttcctagggatgctctaacaaaatactacaactaagtggctcaaaacaacagaaatgtctgtctgtctcacactgctggcagccagatgtcgtgtgtgtgtgtgtgtgtgtgtgtgtgtgtgtgtgtgtgtgtgtgtgtagatcttctctagagctctaaaataggcttctcacagcttcccgaagtgtttggtccttcctgagatggaagatggttggtcatcttcattcagtgtctgcatttcatcgcccctctgtgtgtgtctctttttacaaggacactgtgggtcattcacctggggcctccttacctcagtgacctcaatgaccctggaaatcattctctctaaaaagacaacacttggaggtacggggactactatagcatgttatagaaagctatgtcacgtgactttcgtgagaataacagcgttataaacctcatgagcttcaagacttagattatgatacctgtactacattaggtaactgcttaacgttaaagtgtcagctgaggcaccttaaagggttacagtgtatctgagcaaacaccagttgttgctgggcagctccaacccctgtgtggcccaggcacttcaggtgggaactaactcattagggaggcataaacgccaagccaacgtgattggttacagctccacagctgtttatttggcctcttcccattggagagtccctagttatataaaattttggctgccatggattggttgagtttgttttggttttctttaatatacaaatctatttaaagtagtttatatcacatataataaataaataaatcttttttaaaaaaacttttttgttttcaaatcatgcaaagctaaggtcacgtaggagaaagaccatgactgtgtgcctttttatgctttgggtctggtctccattttaatactttaacactatggtcagttactgatacagaaaatcaaatccaagccaagataa

SEQ ID NO: 21 - DNA, 1237 bp, Mus sp.
  CDS (1)..(1236)
ttatcttggcttggatttgattttctgtatcagtaactgaccatagtgttaaagtattaaaatggagaccagacccaaagcataaaaaggcacacagtcatggtctttctcctacgtgaccttagctttgcatgatttgaaaacaaaaaagtttttttaaaaaagatttatttatttattatatgtgatataaactactttaaatagatttgtatattaaagaaaaccaaaacaaactcaaccaatccatggcagccaaaattttatataactagggactctccaatgggaagaggccaaataaacagctgtggagctgtaaccaatcacgttggcttggcgtttatgcctccctaatgagttagttcccacctgaagtgcctgggccacacaggggttggagctgcccagcaacaactggtgtttgctcagatacactgtaaccctttaaggtgcctcagctgacactttaacgttaagcagttacctaatgtagtacaggtatcataatctaagtcttgaagctcatgaggtttataacgctgttattctcacgaaagtcacgtgacatagctttctataacatgctatagtagtccccgtacctccaagtgttgtctttttagagagaatgatttccagggtcattgaggtcactgaggtaaggaggccccaggtgaatgacccacagtgtccttgtaaaaagagacacacacagaggggcgatgaaatgcagacactgaatgaagatgaccaaccatcttccatctcaggaaggaccaaacacttcgggaagctgtgagaagcctattttagagctctagagaagatctacacacacacacacacacacacacacacacacacacacacacacacacgacatctggctgccagcagtgtgagacagacagacatttctgttgttttgagccacttagttgtagtattttgttagagcatccctaggaagctagagcgctcctcttactctacaccgggtacatctcaggagtcccccatggatggatggtggaagctgcagactatcagcccctgtgtgtcctgtttttctgtattcatttatgcttatgataaagtgtaacttgtaaattaggcaaaggaagaaataaacaactactaatagtaaataactcacattagaatgattataatatactgtgtaactttgtaagcaatatactgcaataaatgttttgcgactgggccctccctt

SEQ ID NO: 22 - protein, 393 aa, Mus sp.
KGGPSRKTFIAVYCLQSYTVYYNHSNVSYLLLVVVYFFLCLIYKLHFIISINEYRKTGHTGADSLQLPPSIHGGLLRCTRCRVRGALLPRDALTKYYNVAQNNRNVCLSHTAGSQMSCVCVCVCVCVCVCVCVDLLSSKIGFSQLPEVFGPSDGRWLVIFIQCLHFIAPLCVSLFTRTLWVIHLGPPYLSDLNDPGNHSLKDNTWRYGDYYSMLKAMSRDFRENNSVINLMSFKTIMIPVLHVTARSVSGTLKGYSVSEQTPVVAGQLQPLCGPGTSGGNLIREATPSQRDWLQLHSCLFGLFPLESPLYKILAAMDWLSLFWFSLIYKSISSLYHIINKSFLKKLFCFQIMQSGHVGERPLCAFLCFGSGLHFNTLTLWSVTDTENQIQAKI

SEQ ID NO: 23 - protein, 389 aa, Mus sp.
REGPVAKHLLQYIAYKVTQYIIIILMVIYYLFISSFAFTSYTLSAMNTEKQDTQGLIVCSFHHPSMGDSDVPGVEEERSSFLGMLQNTTTKWLKTTEMSVCLTLLAARCRVCVCVCVCVCVCVCVIFSRALKASHSFPKCLVLPEMEDGWSSSFSVCISSPLCVCLFLQGHCGSFTWGLLTSVTSMTLEIILSKKTTLGGTGTTIACYRKLCHVTFVRITALTSASRLRLYLYYIRLLNVKVSAEAPRVTVYLSKHQLLLGSSNPCVAQALQVGTNSLGRHKRQANVIGYSSTAVYLASSHWRVPSYIKFWLPWIGVCFGFLYTNLFKVVYITYNKINLFKNFFVFKSCKAKVTEKDHDCVPFYALGLVSILILHYGQLLIQKIKSKPR

SEQ ID NO: 24 - protein, 389 aa, Mus sp.
GRAQSQNIYCSILLTKLHSILSFCELFTISSCLFLPLPNLQVTLYHKHKIQKNRTHRGSAASTIHPWGTPEMYPVSKRSALASGCSNKILQLSGSKQQKCLSVSHCWQPDVVCVCVCVCVCVCVCVCRSSLELNRLLTASRSVWSFLRWKMVGHLHSVSAFHRPSVCVSFYKDTVGHSPGASLPQPQPWKSFSLKRQHLEVRGLLHVIESYVTLSEQRYKPHELQDLDYDTCTTLGNCLTLKCQLRHLKGLQCIANTSCCWAAPTPVWPRHFRWELTHGGINAKPTLVTAPQLFIWPLPIGESLVINFGCHGLVEFVLVFFNIQIYLKFISHIINKIFFKKTFLFSNHAKLRSRRRKTMTVCLFMLWVWSPFYFNTMVSYYRKSNPSQD

SEQ ID NO: 25 - protein, 387 aa, Mus sp.
LSWLGFDFLYQLTIVLKYNGDQTQSIKRHTVMVFLLRDLSFAFENKKVFLKKIYLFIICDINYFKICILKKTKTNSTNPWQPKFYITRDSPMGRGQINSCGAVTNHVGLAFMPPVSSHLKCLGHTGVGAAQQQLVFAQIHCNPLRCLSHFNVKQLPNVVQVSSKSSSGLRCYSHESHVTLSITCYSSPRTSKCCLFRENDFQGHGHGKEAPGEPTVSLKETHTEGRNADTERPTIFHLRKDQTLREAVRSLFSSREDLHTHTHTHTHTHTHTHTTSGCQQCETDRHFCCFEPLSCSILLEHPEARALLLLYTGYISGVPHGWMVEAADYQPLCVLFFCIHLCLSVTCKLGKGRNKQLLIVNNSHNDYNILCNFVSNILQMFCDWALP

SEQ ID NO: 26 - protein, 389 aa, Mus sp.
YLGLDLIFCISNPCSIKMETRPKAKGTQSWSFSYVTLALHDLKTKKFFKRFIYLLYVITTLNRFVYRKPKQTQPIHGSQNFILGTLQWEEAKTAVELPITLAWRLCLPNELVPTSAWATQGLELPSNNWCLLRYTVTLGASADTLTLSSYLMYRYHNLSLEAHEVYNAVILTKVTHSFLHAIVVPVPPSVVFLERMISRVIEVTEVRRPQVNDPQCPCKKRHTQRGDEMQTLNEDDQPSSISGRTKHFGKLEAYFRALEKIYTHTHTHTHTHTHTHTRHLAASSVRQTDISVVLSHLVVVFCSIPRKLERSSYSTPGTSQESPMDGWWKLQTISPCVSCFSVFIYAYDKVLVNAKEEINNYITHIRMIIIYCVTLAIYCNKCFATGPSL

SEQ ID NO: 27 - protein, 379 aa, Mus sp.
ILAWIFSVSVTDHSVKVLKWRPDPKHKKAHSHGLSPTPLCMIKQKSFFKKDLFIYYMYKLLIDLYIKENQNKLNQSMAAKILYNGLSNGKRPNKQLWSCNQSRWLGVYASLMSFPPEVPGPHRGWSCPATTGVCSDTLPFKVPQLTLRAVTCSTGIIIVLKLMRFITLLFSRKSRDIAFYNMLSPYLQVLSFREFPGSLRSLRGGPRMTHSVLVKRDTHRGAMKCRHMKMTNHLPSQEGPNTSGSCEKPILELRRSTHTHTHTHTHTHTHTHDIWLPAVDRQTFLLFATLYFVRASLGSSAPLTLHRVHLRSPPWMDGGSCRLSAPVCPVFLYSFMLMIKCNLIRQRKKTTTNSKLTLELYTVLCKQYTAINVLRLGPP